(19) United States
(12) Patent Application Publication
(10) Pub. No.: US 2024/0207329 A1
(43) Pub. Date: Jun. 27, 2024

(54) METHODS OF TREATING ADENOCARCINOMA WITH HUMAN MICROBIOTA DERIVED N-ACYL AMIDES

(71) Applicants: ICAHN SCHOOL OF MEDICINE AT MOUNT SINAI, New York, NY (US); ROCKEFELLER UNIVERSITY, New York, NY (US)

(72) Inventors: Louis J. Cohen, New York, NY (US); Sean Brady, New York, NY (US); Scott L. Freidman, New York, NY (US)

(21) Appl. No.: 18/556,218

(22) PCT Filed: Apr. 19, 2022
(86) PCT No.: PCT/US2022/025323

§ 371 Date: Oct. 19, 2023

Related U.S. Application Data
(60) Provisional application No. 63/178,887, filed on Apr. 23, 2021.

(51) Int. Cl. A61K 35/74; A61K 31/164; A61K 38/45; A61P 35/00

(52) U.S. Cl. CPC A61K 35/74; A61K 31/164; A61K 38/45; A61P 35/00; C12Y 203/01

(57) ABSTRACT

The presently claimed and described technology provides methods of treating adenocarcinoma in a subject by administering a genetically engineered cell expressing a human microbial N-acyl synthase (hm-NAS) gene, an hm-NAS gene, an N-acyl amide, or compositions thereof.

### RELATED APPLICATIONS

The present patent application claims the priority benefit of U.S. Provisional Patent Application Ser. No. 63/178,887, filed Apr. 23, 2021, the content of which is hereby incorporated by reference in its entirety into this disclosure.

### STATEMENT REGARDING FEDERALLY SPONSORED RESEARCH OR DEVELOPMENT

This invention was made with government support under grant no. DK109287 and grant no. 1R03DK124742-01 awarded by the National Institutes of Health. The Government has certain rights in the invention.

### BACKGROUND

Long-chain N-acyl amides are an important class of human signaling molecules that help to control immunity and behavior and metabolism, among other aspects of human physiology (Hanus et al., 2014, BioFactors 40:381-8). N-acyl amides are able to regulate such diverse human cellular functions due, in part, to their ability to interact with G protein-coupled receptors (GPCRs). GPCRs are the largest family of membrane receptors in eukaryotes and are likely to be key mediators of host-microbial interactions in the human microbiome. The importance of GPCRs to human physiology is reflected in that they are the most common targets of therapeutically approved small molecule drugs. Further, the GPCRs with which human N-acyl amides interact are involved in diseases including cancer (Carri et al., 2015, Nat Rev Endocrinol 12: 133-43; Pacher et al., 2013, FEBS J 280: 1918-43). With numerous possible combinations of amine head groups and acyl tails, long-chain N-acyl amides represent a large and functionally diverse class of microbiota-encoded GPCR-active signaling molecules.

Existing strategies for treating diseases associated with the microbiome, such as metabolic liver diseases including cancer, are not believed to address the dysfunction of the host-microbial interactions that are likely to be part of the disease pathogenesis. Bacteria engineered to deliver bioactive small molecules produced by the human microbiota have the potential to help address diseases of the microbiome by modulating the native distribution and abundance of these metabolites. Regulation of GPCRs by microbiota-derived N-acyl amides is a particularly noteworthy therapeutic strategy for the treatment of human diseases because GPCRs have been extensively validated as therapeutic targets.

Currently, there are no N-acyl amide biotherapeutics available for the treatment of metabolic liver disease and liver cancer, and the present disclosure addresses this unmet need in the art.

### BRIEF SUMMARY

In one aspect, the disclosure provides a method of treating adenocarcinoma in a subject by administering to the subject an N-acyl amide having Formula (1):

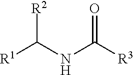

wherein R¹ is selected from the group consisting of carboxylate and CH₂OH; R² is selected from the group consisting of H, (C₃-C₄) alkyl-NH₃⁺, (C₃-C₄)alkyl-NH2, C₂ alkyl-C(═O)NH₂, CH₂OH, and methyl; and R³ is selected from the group consisting of (C₉-C₁₈)alkyl, (C₉-C₁₈)alkenyl, wherein the (C₉-C₁₈)alkyl and (C₉-C₁₈)alkenyl are optionally substituted.

In some aspects, Formula (1) of the N-acyl amide is represented by one of Formulae (2)-(6):

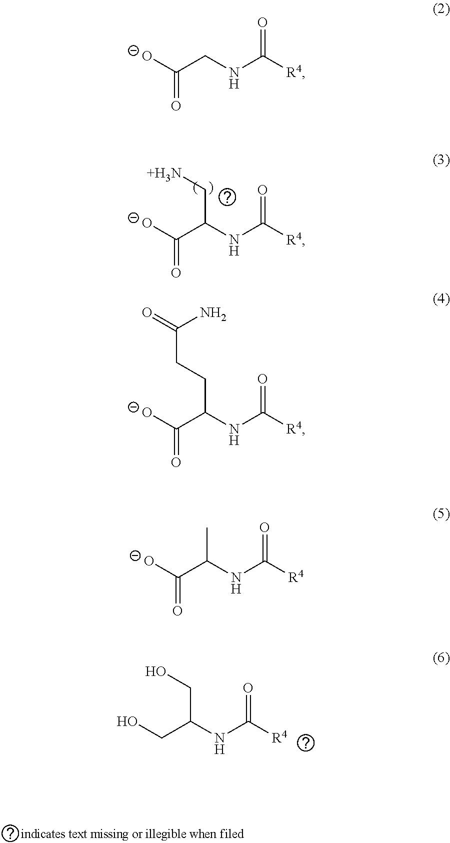

wherein R⁴ is selected from the group consisting of (C₉-C₁₈)alkyl, (C₉-C₁₈)alkenyl, wherein the (C₉-C₁₈)alkyl and (C₉-C₁₈)alkenyl are optionally substituted; and n is 3 or 4. In some aspects of the method, Formulae (2)-(6) are represented by Formulae (7)-(11):

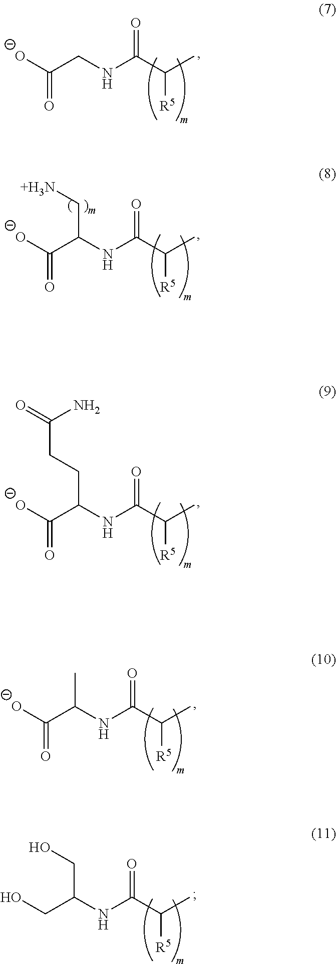

wherein R⁵ is independently selected from the group consisting of H and —OH; and m is an integer from 8 to 17.

In some aspects of the method, Formulae (2)-(6) are represented by Formulae (12)-(16):

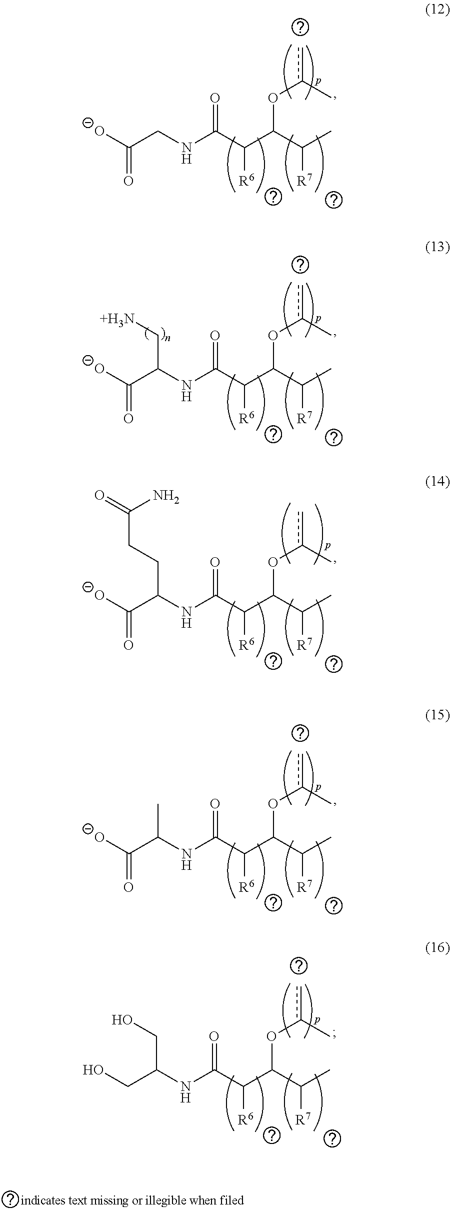

wherein R6, R7, and R8 are independently selected from the group consisting of H, —OH, and ═O; m is an integer from 1 to 5; n is an integer from 2 to 15; p is an integer from 8 to 18; and q is an integer from 3 to 4.

In some aspects of the method, the N-acyl amide is selected from the group consisting of:

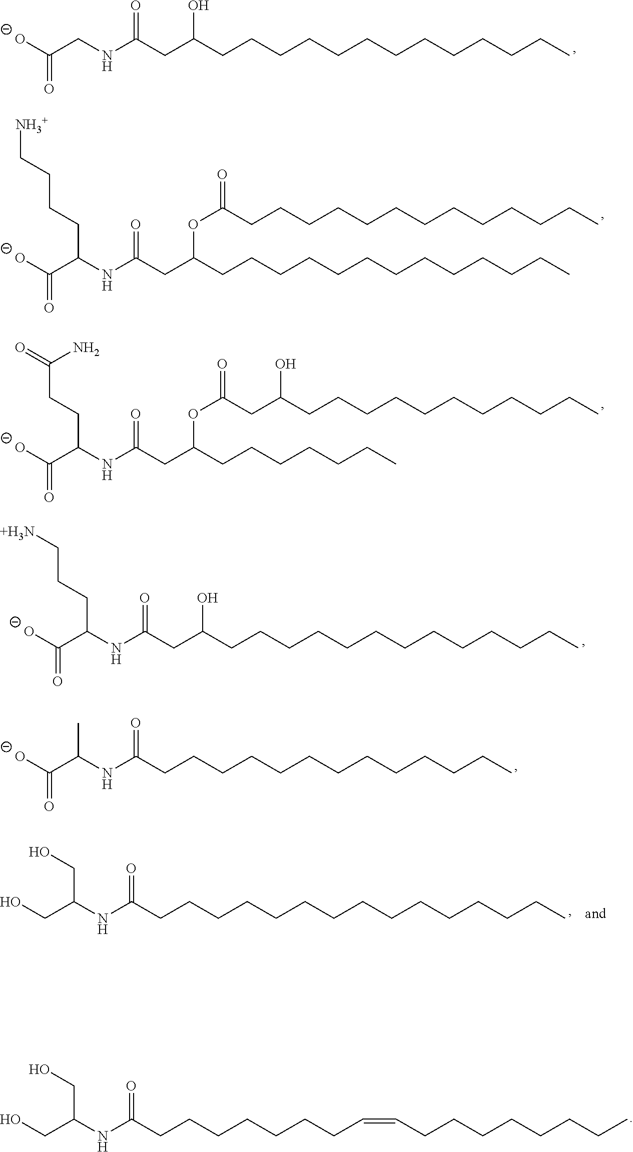

In some aspects of the method, the N-acyl amide is N-acyl serinol or, more specifically, N-oleoyl serinol.

In some aspects of the method, the adenocarcinoma can be found in the digestive system of the subject. More specifically, the adenocarcinoma can be found in the liver, pancreas, small intestine, large intestine, colon, or stomach of the subject. In some aspects, the adenocarcinoma is hepatocellular carcinoma.

In another aspect, the disclosure provides a method of treating adenocarcinoma in a subject by administering to the subject a composition comprising at least one of a genetically engineered cell expressing a human microbial N-acyl synthase (hm-NAS) gene, an hm-NAS gene, or an N-acyl amide.

In some aspects of the method, the genetically engineered cell encodes an N-acyl synthase polypeptide that catalyzes synthesis of an N-acyl amide. In certain aspects, the genetically engineered cell is a non-pathogenic bacterial cell, such as but not limited to, E. coli.

In some aspects of the method, the hm-NAS gene is selected from the group consisting of EFI7261; EHB91285; EEK17761; EEY82825; EHP49568; EHG23013; EFA42931; EFL47029; EH075052; ADK95845; EFV04460; EHH01788; EDY97076; CBW20928; EDS14876; ED052243; CBK67812; AC109609; ABV66681; EHT12133; EFE54303; EFE94777; EER56350; EET45812; ACS62992; BAH33083; EFG73978; CAW29482; EFH13337; EGP09383; EEV22085; EEY94333; EFF83269; CAP01857; EGP10046; EFK33376; EEK14630; EFS97491; CBK85930; EHM48796; EEK89350; EHL05550; EFV76279; GL883582; R6A3N1_9BACT/51-156; R6EH40_9BACT/51-155; R7PBT6_9BACT/52-156; R7NN97 9BACE/51-155; AOAOC3RD59_9PORP/51-157; A6L081 BACV8/51-155; A6LEV2_PARD8/51-155; D41Mll 9BACT/57-158; D5EVS3_PRER2/52-157; D6D060 9BACE/51-155; E6SVIO_BACT6/51-155; CBK67812_CBK67812.l_Bacteroides_xylanisolvens_XB1A_hypothetical_protein; ENA_CBW20928_CBW20928.l_Bacteroides_fragilis_638R_putative_hemolysin_A; ENA_ED052243 ED052243.l_Bacteroides_uniformis_ATCC_8492_hemolysin; ENA_EDSl 4876_EDS 14876.l_Bacteroides_stercoris_ATCC_43183_hemolysin_; ENA_EDY97076_EDY97076.l_Bacteroides_plebeius_DSM_1 7135 hemolysin_; ENA_EEY82825_EEY82825.l_Bacteroides_sp._2_1_33B_hemolysin_; ENA_EFV04460_EFV04460.l_Prevotella_salivae_DSM_15606_hemolysin_; ENA_EHB91285_EHB91285.1 Alistipes_indistinctus_YIT_12060_hypothetical_protein_; ENA_EHH01788_EHH01 788.l_Paraprevotella_clara_YIT 1 840_hemolysin; ENA_EHP49568_EHP49568.l_Odoribacter_laneus_YIT_12061_hypothetical_protein; 13YLB0_ALIFI/56-157; Q5LII1_BACFN/51-155; Q8A247 BACTN/51-155; R5C642 9BACE/51-155; R5FQF1_9BACT/53-157; R51942_9PORP/51-156; R5JGR8 9BACE/51-155; R5KD71 9BACT/52-157; R5MMX8 9BACE/51-155; R5NZI1 9BACT/51-155; R5UEV5 9BACE/51-155; R5UP15_9PORP/51-157; R5VW07_9BACE/51-155; R6B4U0_9BACT/52-156; R6BXV9 9BACT/52-157; R6DH15 9BACE/51-155; R6FKP1 9BACE/51-155; R6FUQ8_9BACT/52-158; R6KTM3 9BACE/51-155; R6LNJ9_9BACE/51-154; R6MX16 9BACE/51-155; R6QE29_9BACT/52-157; R6S950_9BACE/51-155; R6SC61_9BACE/51-155; R6VUA1_9BACT/56-157; R6XGV7 9BACT/52-157; R6YIB5_9BACE/51-155; R7DDR3 9PORP/51-155; R7EIP8_9BACE/51-155; R7F021_9BACT/51-157; R7HSG0_9BACT/37-143; R7IYP9_9BACT/59-165; R7JHM4_9BACT/51-152; E6K481 9BACT/52-156; ENA_ADK95 845 ADK95845.l_Prevotella_melaninogenica_ATCC_25 845 hemolysin_; ENA_EFil 7261_EF!l 7261.l_Bacteroidetes_oral_taxon_274_str._F0058_hemolysin; ENA_EHG23013_EHG23013.l_Alloprevotella_rava_F0323_hypothetical_protein; ENA_EHO7 5052_EHO75052.l_Prevotella_micans_F0438_hypothetical_protein; F2KX19_PREDF/64-168; F903S1_PREDD/52-156 1; 11 YUM9 PREl7/53-157; Q7MTR9_PORGV53-158; R5CSR0_9BACT/52-157; R5GFN8_9BACT/51-155; R5Q4D6_9BACT/52-157; R6W2Q2_9BACT/52-156; R7CYB8 9BACE/51-155; WOEP20 9PORP/51-155; C7M608_CAPOD/352-453; ENA_EEK14630_EEK14630.l_Capnocytophaga_gingivalis_ATCC_33624_Acyltransferase_; ENA_EFS97491_EFS97491.l_Capnocytophaga_ochracea_F0287_Acyltransferase; F9YU78_CAPCC/351-452; H1Z9S5 MYROD/346-447 ENA_EFA42931_EFA4293 l.l_Prevotella_bergensis_DSM_1 7361_hemolysin; A0A095ZG93 9BACT/52-156; E7RNE3 9BACT/52-156; ENA_EEK1 7761_EEK1 7761.l_Porphyromonas_uenonis_60-3_hemolysin_; ENA EFIA7029 EFL47029.1 Prevotella_disiens_FB035-09AN_hemolysin_; F4KL89_PORAD/55-160; 14Z8L9 9BACT/52-156; R6CE12 9BACE/51-155; R6XAK6_9BACT/52-156 ENA EHL05550 EHL05550.l_Desulfitobacterium_hafniense_DP7 aminotransferase class_V; ENA_EFV76279 EFV76279.l_Bacillus_sp._2_A_57 CT2_serinepyruvate_arninotransferase; A6T596_KLEP7/322-423; D8MWX6_ERWBE/367-468; ENA_EFE94777 EFE94 777.l_Serratia_odorifera_DSM_45 82_Acyltransferase; Q6CZN2_PECAS/322-423; AOAOB5CH45_NEIEG/32-132; E5UJR0_NEIMU/32-132; ENA_EET 45 812_EET 45 812.l_Neisseria_sicca_ATCC_29256_hypothetical_protein; ENA_ACI09609_ACI09609.l_Klebsiella_pneumoniae_342_conserved_hypothetical_protein; A4W746 ENT38/322-423; ENA_CBK85930_CBK85930.l_Enterobacter_cloacae_subsp._cloacae_NCTC_9394_Putative_h emolysin_; ENA_EFE54303_EFE54303.l_Providencia_rettgeri_DSM_ll31_Acyltransferase; ENA_EHM48796_EHM48796.l_Yokenella_regensburgei_ATCC_43003_Acyltransferase; F9ZAJ4_ODOSD/341-443; G9Z3T1 9ENTR/322-423; R5UYM1_9PORP/338-439; ENA_ACS62992_ACS62992.l_Ralstonia_pickettii_12D_conserved_hypothetical_protein_; ENA_CAW29482_CAW29482.l_Pseudomonas_aeruginosa_LESB58_putative_hemolysin_; AOA089UDH2_9ENTR/323-424; E6WAC8_PANSA/322-423; ENA_EHT12133_EH T12133.l_Raoultella_omithinolytica_10-5246_hypothetical_protein; G7LV45_9EN TR/322-423; ENA_EER56350_EER56350.l_N eisseria_flavescens_SK1 1 4_hypothetical_protein_; AOA077KL19 9FLAO/353-454; A7MLT3_CROS8/322-423; ENA_EFK33376_EF K33376.l_Chryseobacterium_gleum_ATCC_35910_Acyltransferase_; and ENA_CAPOl 857 CAP01857.2_Acinetobacter_baumannii_SDF conserved_hypothetical_protein_.

In some aspects of the method, the hm-NAS gene is N-acyl serinol synthase.

In some aspects of the method, the N-acyl amide has Formula (1):

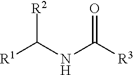

wherein R¹ is selected from the group consisting of carboxylate and CH₂OH; R² is selected from the group consisting of H, (C₃-C₄) alkyl-NH₃⁺, (C₃-C₄)alkyl-NH₂, C₂ alkyl-C(═O)NH₂, CH₂OH, and methyl; and R³ is selected from the group consisting of (C₉-C₁₈)alkyl, (C₉-C₁₈)alkenyl, wherein the (C₉-C₁₈)alkyl and (C₉-C₁₈)alkenyl are optionally substituted.

In some aspects of the method, Formula (1) of the N-acyl amide is represented by one of Formulae (2)-(6):

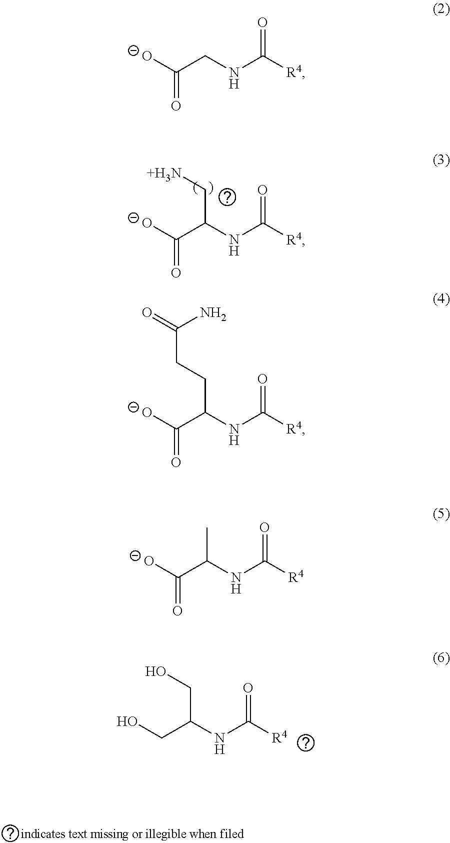

wherein R⁴ is selected from the group consisting of (C₉-C₁₈)alkyl, (C₉-C₁₈)alkenyl, wherein the (C₉-C₁₈)alkyl and (C₉-C₁₈)alkenyl are optionally substituted; and n is 3 or 4.

In some aspects of the method, Formulae (2)-(6) are represented by Formulae (7)-(11):

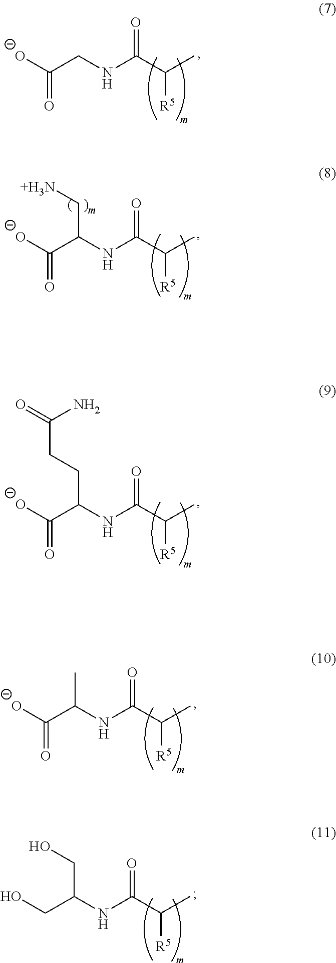

wherein R⁵ is independently selected from the group consisting of H and —OH; and m is an integer from 8 to 17.

In some aspects of the method, Formulae (2)-(6) are represented by Formulae (12)-(16):

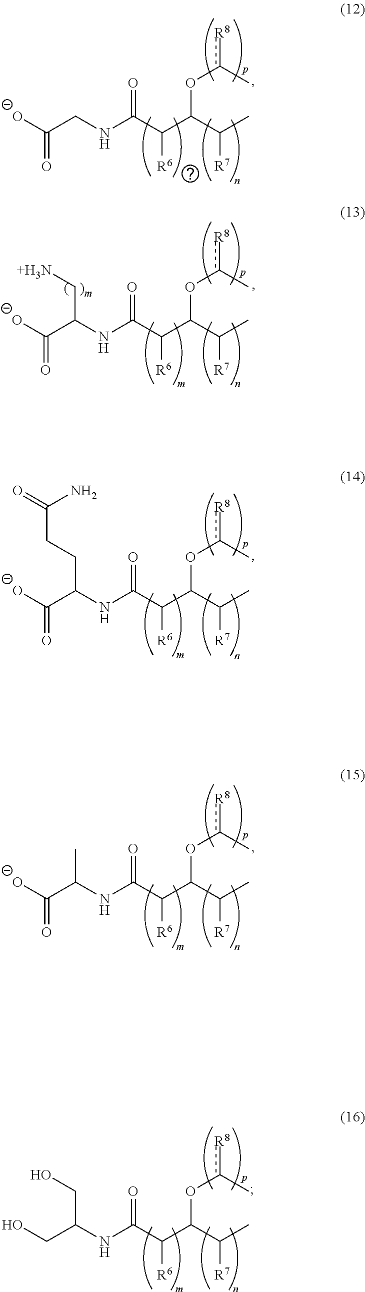

wherein R⁶, R⁷, and R⁸ are independently selected from the group consisting of H, —OH, and ═O; m is an integer from 1 to 5; n is an integer from 2 to 15; p is an integer from 8 to 18; and q is an integer from 3 to 4.

In some aspects of the method, the N-acyl amide is selected from the group consisting of:

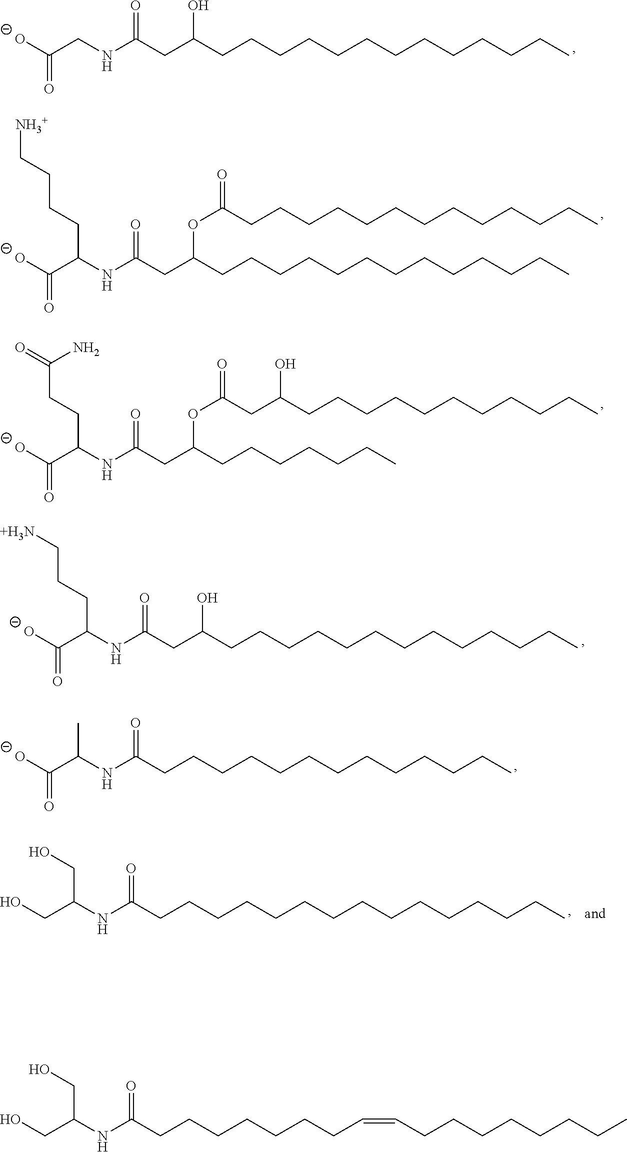

In some aspects of the method, the N-acyl amide is N-acyl serinol or, more specifically, N-oleoyl serinol.

In another aspect of the method, the composition is administered in a therapeutically effective amount, and/or the composition further comprises a pharmaceutically acceptable carrier, diluent, buffer, or excipient.

In some aspects of the method, the adenocarcinoma can be found in the digestive system of the subject. More specifically, the adenocarcinoma can be found in the liver, pancreas, small intestine, large intestine, colon, or stomach of the subject. In some aspects, the adenocarcinoma is hepatocellular carcinoma.

In yet another aspect, the disclosure provides a method of treating liver cancer in a subject by administering to the subject a composition comprising at least one of a genetically engineered cell expressing a human microbial N-acyl synthase (hm-NAS) gene, an hm-NAS gene, or an N-acyl amide.

In some aspects of the method, the genetically engineered cell encodes an N-acyl synthase polypeptide that catalyzes synthesis of an N-acyl amide.

In some aspects of the method, the genetically engineered cell is a non-pathogenic bacterial cell, such as but not limited to, E. coli.

In some aspects of the method, the hm-NAS gene is selected from the group consisting of EFI7261; EHB91285; EEK17761; EEY82825; EHP49568; EHG23013; EFA42931; EFL47029; EH075052; ADK95845; EFV04460; EHH01788; EDY97076; CBW20928; EDS14876; ED052243; CBK67812; AC109609; ABV66681; EHT12133; EFE54303; EFE94777; EER56350; EET45812; ACS62992; BAH33083; EFG73978; CAW29482; EFH13337; EGP09383; EEV22085; EEY94333; EFF83269; CAP01857; EGP10046; EFK33376; EEK14630; EFS97491; CBK85930; EHM48796; EEK89350; EHL05550; EFV76279; GL883582; R6A3N1_9BACT/51-156; R6EH40_9BACT/51-155; R7PBT6_9BACT/52-156; R7NN97 9BACE/51-155; AOAOC3RD59_9PORP/51-157; A6L081 BACV8/51-155; A6LEV2_PARD8/51-155; D41Mll 9BACT/57-158; D5EVS3_PRER2/52-157; D6D060 9BACE/51-155; E6SVIO_BACT6/51-155; CBK67812_CBK67812.l_Bacteroides_xylanisolvens_XB1A_hypothetical_protein; ENA_CBW20928_CBW20928.l_Bacteroides_fragilis_638R_putative_hemolysin_A; ENA_ED052243 ED052243. l_Bacteroides_uniformis_ATCC_8492_hemolysin; ENA_EDS1 4876_EDS 14876.l_Bacteroides_stercoris_ATCC_43183_hemolysin_; ENA_EDY97076_EDY97076.l_Bacteroides_plebeius_DSM_1 7135 hemolysin_; ENA_EEY82825_EEY82825.l_Bacteroides_sp._2_1_33B_hemolysin_; ENA_EFV04460_EFV04460.l_Prevotella_salivae_DSM_15606_hemolysin_; ENA_EHB91285_EHB91285.1 Alistipes_indistinctus_YIT_12060_hypothetical_protein_; ENA_EHH01788_EHH01 788.l_Paraprevotella_clara_YIT 11 840_hemolysin; ENA_EHP49568_EHP49568.l_Odoribacter_laneus_YIT_12061_hypothetical_protein; 13YLB0_ALIFI/56-157; Q5LIIl_BACFN/51-155; Q8A247 BACTN/51-155; R5C642 9BACE/51-155; R5FQF1_9BACT/53-157; R51942_9PORP/51-156; R5JGR8 9BACE/51-155; R5KD71 9BACT/52-157; R5MMX8 9BACE/51-155; R5NZI1 9BACT/51-155; R5UEV5 9BACE/51-155; R5UP15_9PORP/51-157; R5VW07_9BACE/51-155; R6B4U0_9BACT/52-156; R6BXV9 9BACT/52-157; R6DH15 9BACE/51-155; R6FKP1 9BACE/51-155; R6FUQ8_9BACT/52-158; R6KTM3 9BACE/51-155; R6LNJ9_9BACE/51-154; R6MX16 9BACE/51-155; R6QE29_9BACT/52-157; R6S950_9BACE/51-155; R6SC61_9BACE/51-155; R6VUA1_9BACT/56-157; R6XGV7 9BACT/52-157; R6YIB5_9BACE/51-155; R7DDR3 9PORP/51-155; R7EIP8_9BACE/51-155; R7F021_9BACT/51-157; R7HSG0_9BACT/37-143; R7IYP9_9BACT/59-165; R7JHM4_9BACT/51-152; E6K481 9BACT/52-156; ENA_ADK95 845 ADK95845.l_Prevotella_melaninogenica_ATCC_25 845 hemolysin_; ENA_EFil 7261_EF!1 7261.l_Bacteroidetes_oral_taxon_274_str._F0058_hemolysin; ENA_EHG23013_EHG23013.l_Alloprevotella_rava_F0323_hypothetical_protein; ENA_EHO7 5052_EHO75052.l_Prevotella_micans_F0438_hypothetical_protein; F2KX19_PREDF/64-168; F903Sl_PREDD/52-156 1; 11 YUM9 PREl7/53-157; Q7MTR9_PORGV53-158; R5CSR0_9BACT/52-157; R5GFN8_9BACT/51-155; R5Q4D6_9BACT/52-157; R6W2Q2_9BACT/52-156; R7CYB8 9BACE/51-155; WOEP20 9PORP/51-155; C7M608_CAPOD/352-453; ENA_EEK14630_EEK14630.l_Capnocytophaga_gingivalis_ATCC_33624_Acyltransferase_; ENA_EFS97491_EFS97491.l_Capnocytophaga_ochracea_F0287_Acyltransferase; F9YU78_CAPCC/351-452; H1Z9S5 MYROD/346-447 ENA_EFA42931_EFA4293 l.l_Prevotella_bergensis_DSM_1 7361_hemolysin; AOA095ZG93 9BACT/52-156; E7RNE3 9BACT/52-156; ENA_EEKI 7761_EEK1 7761.l_Porphyromonas_uenonis_60-3_hemolysin_; ENA EFIA7029 EFL47029.1 Prevotella_disiens_FB035-09AN_hemolysin_; F4KL89_PORAD/55-160; 14Z8L9 9BACT/52-156; R6CE12 9BACE/51-155; R6XAK6_9BACT/52-156 ENA EHL05550 EHL05550.l_Desulfitobacterium_hafniense_DP7 aminotransferase class_V; ENA_EFV76279 EFV76279.l_Bacillus_sp._2_A_57 CT2_serinepyruvate_arninotransferase; A6T596_KLEP7/322-423; D8MWX6_ERWBE/367-468; ENA_EFE94777 EFE94 777.l_Serratia_odorifera_DSM_45 82_Acyltransferase; Q6CZN2_PECAS/322-423; AOAOB5CH45_NEIEG/32-132; E5UJR0_NEIMU/32-132; ENA_EET 45 812_EET 45 812.l_Neisseria_sicca_ATCC_29256_hypothetical_protein; ENA_ACI09609_ACI09609.l_Klebsiella_pneumoniae_342_conserved_hypothetical_protein; A4W746 ENT38/322-423; ENA_CBK85930_CBK85930.l_Enterobacter_cloacae_subsp._cloacae_NCTC_9394_Putative_hemolysin_; ENA_EFE54303_EFE54303.l_Providencia_rettgeri_DSM_ll31_Acyltransferase; ENA_EHM48796_EHM48796.l_Yokenella_regensburgei_ATCC_43003_Acyltransferase; F9ZAJ4_ODOSD/341-443; G9Z3T1 9ENTR/322-423; R5UYM1_9PORP/338-439; ENA_ACS62992_ACS62992.l_Ralstonia_pickettii_12D_conserved_hypothetical_protein_; ENA_CAW29482_CAW29482.l_Pseudomonas_aeruginosa_LESB58_putative_hemolysin_; AOA089UDH2_9ENTR/323-424; E6WAC8_PANSA/322-423; ENA_EHT12133_EH T12133.l_Raoultella_omithinolytica_10-5246_hypothetical_protein; G7LV45_9EN TR/322-423; ENA_EER56350_EER56350.l_N eisseria_flavescens_SK1 1 4_hypothetical_protein_; AOA077KL19 9FLAO/353-454; A7MLT3_CROS8/322-423; ENA_EFK33376_EF K33376.l_Chryseobacterium_gleum_ATCC_35910_Acyltransferase_; and ENA_CAPOl 857 CAP01857.2_Acinetobacter_baumannii_SDF conserved_hypothetical_protein_.

In some aspects of the method, the hm-NAS gene is N-acyl serinol synthase.

In some aspects of the method, the N-acyl amide has Formula (1):

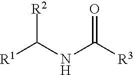

wherein R¹ is selected from the group consisting of carboxylate and CH₂OH; R² is selected from the group consisting of H, (C₃-C₄) alkyl-NH₃⁺, (C₃-C₄)alkyl-NH₂, C₂ alkyl-C(═O)NH₂, CH₂OH, and methyl; and R³ is selected from the group consisting of (C₉-C₁₈)alkyl, (C₉-C₁₈)alkenyl, wherein the (C₉-C₁₈)alkyl and (C₉-C₁₈)alkenyl are optionally substituted.

In some aspects of the method, Formula (1) of the N-acyl amide is represented by one of Formulae (2)-(6):

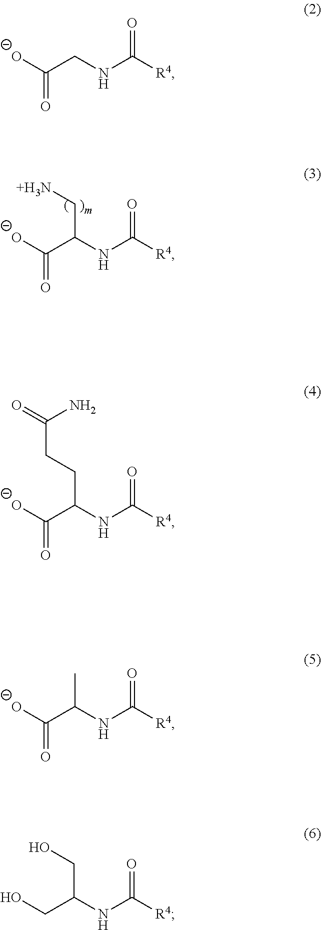

wherein R⁴ is selected from the group consisting of (C₉-C₁₈)alkyl, (C₉-C₁₈)alkenyl, wherein the (C₉-C₁₈)alkyl and (C₉-C₁₈)alkenyl are optionally substituted; and n is 3 or 4.

In some aspects of the method, Formulae (2)-(6) are represented by Formulae (7)-(11):

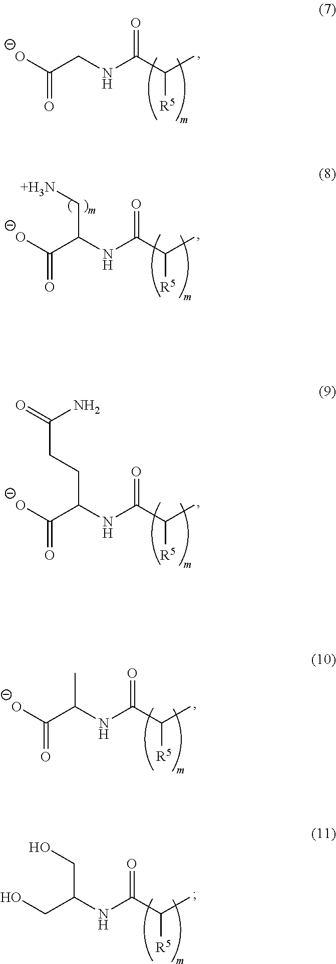

wherein R⁵ is independently selected from the group consisting of H and —OH; and m is an integer from 8 to 17.

In some aspects of the method. Formulae (2)-(6) are represented by Formulae (12)-(16):

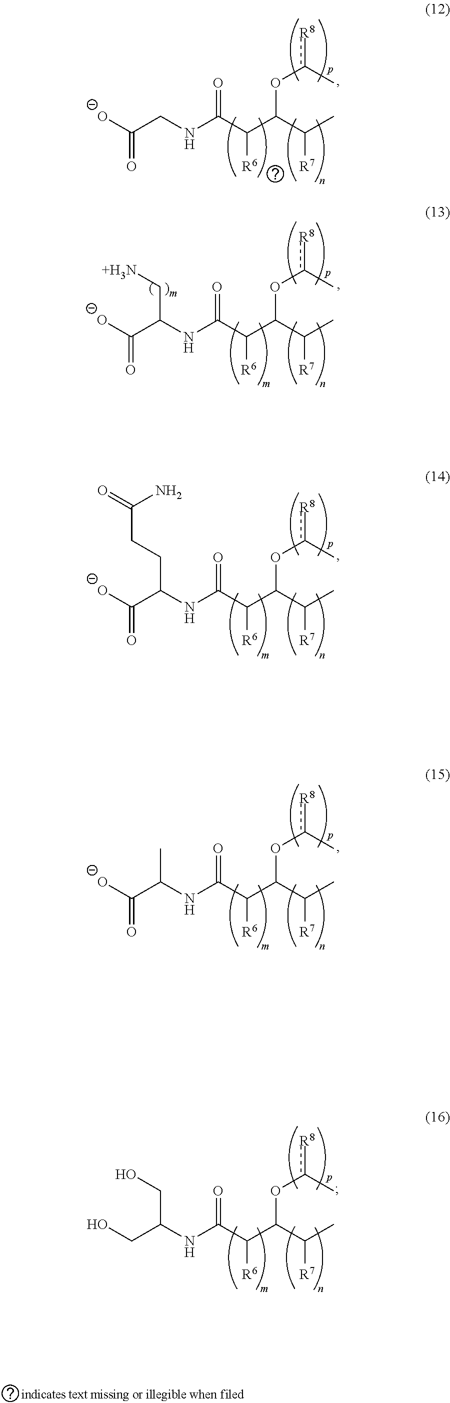

wherein R⁶, R⁷, and R⁸ are independently selected from the group consisting of H, —OH, and ═O; m is an integer from 1 to 5; n is an integer from 2 to 15; p is an integer from 8 to 18; and q is an integer from 3 to 4.

In some aspects of the method, the N-acyl amide is selected from the group consisting of:

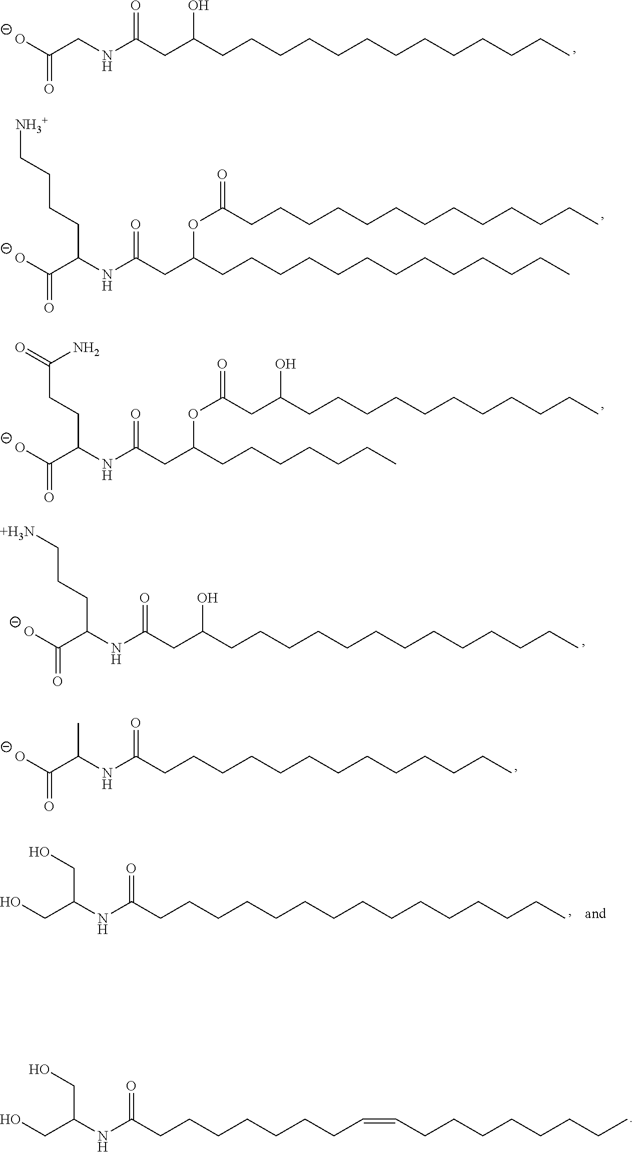

In some aspects of the method, the N-acyl amide is N-acyl serinol or, more specifically, N-oleoyl serinol.

In some aspects of the method, the composition is administered in a therapeutically effective amount, and/or the composition further comprises a pharmaceutically acceptable carrier, diluent, buffer, or excipient.

In some aspects of the method, the liver cancer is hepatocellular carcinoma.

In yet a further aspect, the disclosure provides a method of treating adenocarcinoma in a subject using a live biotherapeutic, the method comprising administering to the subject a composition comprising a genetically engineered cell expressing a human microbial N-acyl synthase (hm-NAS) gene, wherein the hm-NAS gene encodes an N-acyl synthase polypeptide.

In some aspects of the method, the N-acyl synthase polypeptide catalyzes synthesis of an N-acyl amide.

In some aspects of the method, the genetically engineered cell is a non-pathogenic bacterial cell, such as but not limited to, E. coli.

In some aspects of the method, the hm-NAS gene is selected from the group consisting of EFI7261; EHB91285; EEK17761; EEY82825; EHP49568; EHG23013; EFA42931; EFL47029; EH075052; ADK95845; EFV04460; EHH01788; EDY97076; CBW20928; EDS14876; ED052243; CBK67812; AC109609; ABV66681; EHT12133; EFE54303; EFE94777; EER56350; EET45812; ACS62992; BAH33083; EFG73978; CAW29482; EFH13337; EGP09383; EEV22085; EEY94333; EFF83269; CAP01857; EGP10046; EFK33376; EEK14630; EFS97491; CBK85930; EHM48796; EEK89350; EHL05550; EFV76279; GL883582; R6A3N1_9BACT/51-156; R6EH40_9BACT/51-155; R7PBT6_9BACT/52-156; R7NN97 9BACE/51-155; AOAOC3RD59_9PORP/51-157; A6L081 BACV8/51-155; A6LEV2_PARD8/51-155; D41Mll 9BACT/57-158; D5EVS3_PRER2/52-157; D6D060 9BACE/51-155; E6SVIO_BACT6/51-155; CBK67812_CBK67812.l_Bacteroides_xylanisolvens_XB1A_hypothetical_protein; ENA_CBW20928_CBW20928.l_Bacteroides_fragilis_638R_putative_hemolysin_A; ENA_ED052243 ED052243. l_Bacteroides_uniformis_ATCC_8492_hemolysin; ENA_EDS1 4876_EDS 14876.l_Bacteroides_stercoris_ATCC_43183_hemolysin_; ENA_EDY97076_EDY97076.l_Bacteroides_plebeius_DSM_1 7135 hemolysin_; ENA_EEY82825_EEY82825.l_Bacteroides_sp._2_1_33B_hemolysin_; ENA_EFV04460_EFV04460.l_Prevotella_salivae_DSM_15606_hemolysin_; ENA_EHB91285_EHB91285.1 Alistipes_indistinctus_YIT_12060_hypothetical_protein_; ENA_EHH01788_EHH01 788.l_Paraprevotella_clara_YIT 11 840_hemolysin; ENA_EHP49568_EHP49568.l_Odoribacter_laneus_YIT_12061_hypothetical_protein; 13YLB0_ALIFI/56-157; Q5LII1_BACFN/51-155; Q8A247 BACTN/51-155; R5C642 9BACE/51-155; R5FQF1_9BACT/53-157; R51942_9PORP/51-156; R5JGR8 9BACE/51-155; R5KD71 9BACT/52-157; R5MMX8 9BACE/51-155; R5NZI1 9BACT/51-155; R5UEV5 9BACE/51-155; R5UP15_9PORP/51-157; R5VW07_9BACE/51-155; R6B4U0_9BACT/52-156; R6BXV9 9BACT/52-157; R6DH15 9BACE/51-155; R6FKP1 9BACE/51-155; R6FUQ8_9BACT/52-158; R6KTM3 9BACE/51-155; R6LNJ9_9BACE/51-154; R6MX16 9BACE/51-155; R6QE29_9BACT/52-157; R6S950_9BACE/51-155; R6SC61_9BACE/51-155; R6VUA1_9BACT/56-157; R6XGV7 9BACT/52-157; R6YIB5_9BACE/51-155; R7DDR3 9PORP/51-155; R7EIP8_9BACE/51-155; R7F021_9BACT/51-157; R7HSG0_9BACT/37-143; R7IYP9_9BACT/59-165; R7JHM4_9BACT/51-152; E6K481 9BACT/52-156; ENA_ADK95 845 ADK95845.l_Prevotella_melaninogenica_ATCC_25 845 hemolysin_; ENA_EFil 7261_EF!l 7261.l_Bacteroidetes_oral_taxon_274_str._F0058_hemolysin; ENA_EHG23013_EHG23013.l_Alloprevotella_rava_F0323_hypothetical_protein; ENA_EHO7 5052_EHO75052.l_Prevotella_micans_F0438_hypothetical_protein; F2KX19_PREDF/64-168; F903S1_PREDD/52-156 1; 11 YUM9 PREl7/53-157; Q7MTR9_PORGV53-158; R5CSR0_9BACT/52-157; R5GFN8_9BACT/51-155; R5Q4D6_9BACT/52-157; R6W2Q2_9BACT/52-156; R7CYB8 9BACE/51-155; WOEP20 9PORP/51-155; C7M608_CAPOD/352-453; ENA_EEK14630_EEK14630.l_Capnocytophaga_gingivalis_ATCC_33624_Acyltransferase_; ENA_EFS97491_EFS97491.l_Capnocytophaga_ochracea_F0287_Acyltransferase; F9YU78_CAPCC/351-452; H1Z9S5 MYROD/346-447 ENA_EFA42931_EFA4293 l.l_Prevotella_bergensis_DSM_1 7361_hemolysin; A0A095ZG93 9BACT/52-156; E7RNE3 9BACT/52-156; ENA_EEKI 7761_EEK1 7761.l_Porphyromonas_uenonis_60-3_hemolysin_; ENA EFIA7029 EFL47029.1 Prevotella_disiens_FB035-09AN_hemolysin_; F4KL89_PORAD/55-160; 14Z8L9 9BACT/52-156; R6CE12 9BACE/51-155; R6XAK6_9BACT/52-156 ENA EHL05550 EHL05550.l_Desulfitobacterium_hafniense_DP7 aminotransferase class_V; ENA_EFV76279 EFV76279.l_Bacillus_sp._2_A_57 CT2_serinepyruvate_arninotransferase; A6T596_KLEP7/322-423; D8MWX6_ERWBE/367-468; ENA_EFE94777 EFE94 777.l_Serratia_odorifera_DSM_45 82_Acyltransferase; Q6CZN2_PECAS/322-423; AOAOB5CH45_NEIEG/32-132; E5UJR0_NEIMU/32-132; ENA_EET 45 812_EET 45 812.l_Neisseria_sicca_ATCC_29256_hypothetical_protein; ENA_ACI09609_ACI09609.l_Klebsiella_pneumoniae_342_conserved_hypothetical_protein; A4W746 ENT38/322-423; ENA_CBK85930_CBK85930.l_Enterobacter_cloacae_subsp._cloacae_NCTC_9394_Putative_h emolysin_; ENA_EFE54303_EFE54303.l_Providencia_rettgeri_DSM_ll31_Acyltransferase; ENA_EHM48796_EHM48796.l_Yokenella_regensburgei_ATCC_43003_Acyltransferase; F9ZAJ4_ODOSD/341-443; G9Z3T1 9ENTR/322-423; R5UYM1_9PORP/338-439; ENA_ACS62992_ACS62992.l_Ralstonia_pickettii_12D_conserved_hypothetical_protein_; ENA_CAW29482_CAW29482.l_Pseudomonas_aeruginosa_LESB58_putative_hemolysin_; AOA089UDH2_9ENTR/323-424; E6WAC8_PANSA/322-423; ENA_EHT12133_EH T12133.l_Raoultella_omithinolytica_10-5246_hypothetical_protein; G7LV45_9EN TR/322-423; ENA_EER56350_EER56350.l_N eisseria_flavescens_SK1 1 4_hypothetical_protein_; AOA077KL19 9FLAO/353-454; A7MLT3_CROS8/322-423; ENA_EFK33376_EF K33376.l_Chryseobacterium_gleum_ATCC_35910_Acyltransferase_; and ENA_CAPOl 857 CAP01857.2_Acinetobacter_baumannii_SDF conserved_hypothetical_protein_.

In some aspects of the method, the hm-NAS gene is N-acyl serinol synthase.

In some aspects of the method, the N-acyl amide has Formula (1):

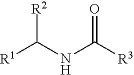

wherein R¹ is selected from the group consisting of carboxylate and CH₂OH; R² is selected from the group consisting of H, (C₃-C₄) alkyl-NH₃⁺, (C₃-C₄)alkyl-NH₂, C₂ alkyl-C(═O)NH₂, CH₂OH, and methyl; and R³ is selected from the group consisting of (C₉-C₁₈)alkyl, (C₉-C₁₈)alkenyl, wherein the (C₉-C₁₈)alkyl and (C₉-C₁₈)alkenyl are optionally substituted.

In some aspects of the method, Formula (1) of the N-acyl amide is represented by one of Formulae (2)-(6):

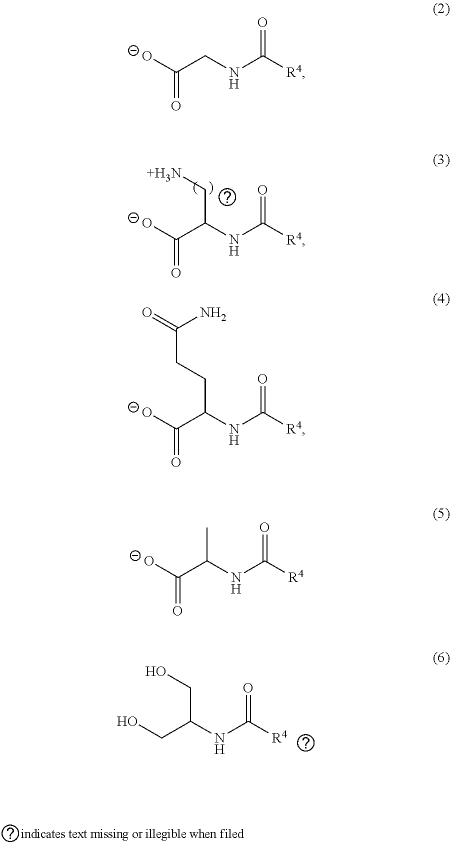

wherein R⁴ is selected from the group consisting of (C₉-C₁₈)alkyl, (C₉-C₁₈)alkenyl, wherein the (C₉-C₁₈)alkyl and (C₉-C₁₈)alkenyl are optionally substituted; and n is 3 or 4.

In some aspects of the method, Formulae (2)-(6) are represented by Formulae (7)-(11):

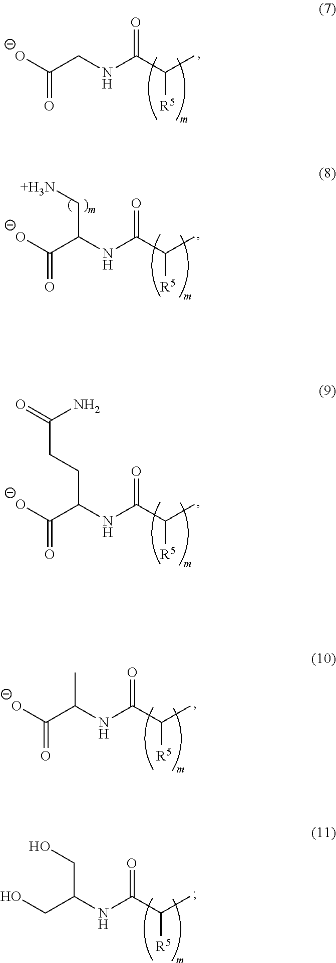

wherein R⁵ is independently selected from the group consisting of H and —OH; and m is an integer from 8 to 17.

In some aspects of the method, Formulae (2)-(6) are represented by Formulae (12)-(16):

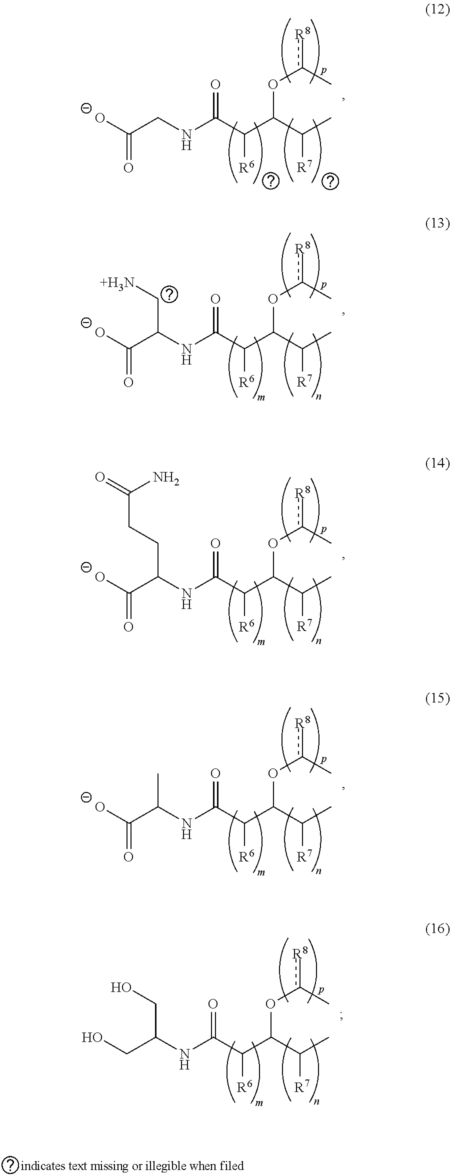

wherein R⁶, R⁷, and R⁸ are independently selected from the group consisting of H, —OH, and ═O; m is an integer from 1 to 5; n is an integer from 2 to 15; p is an integer from 8 to 18; and q is an integer from 3 to 4.

In some aspects of the method, the N-acyl amide is selected from the group consisting of:

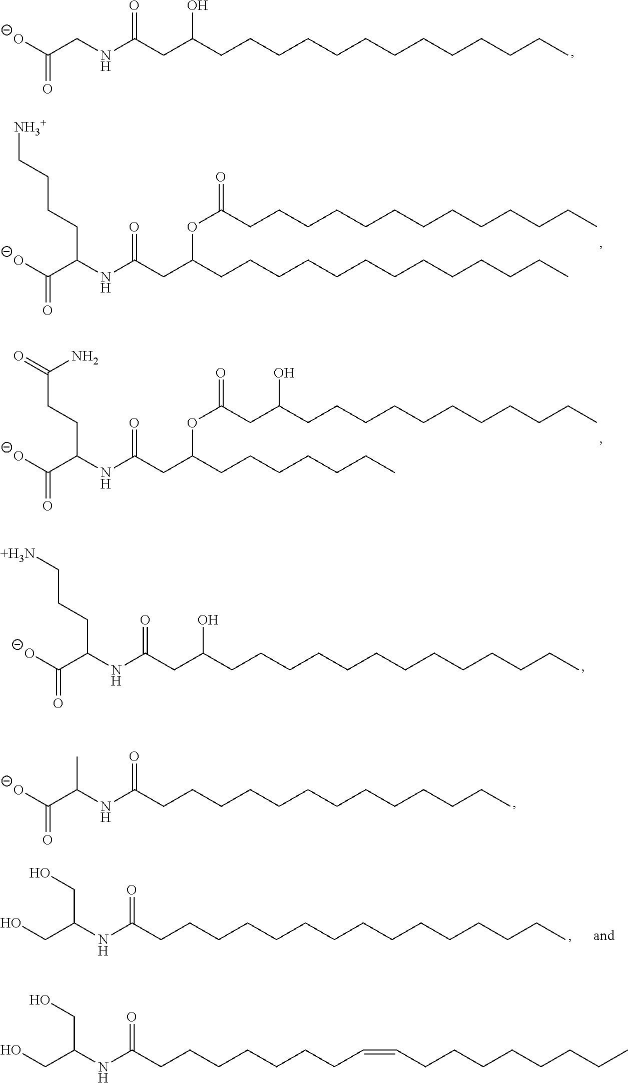

In some aspects of the method, the N-acyl amide is N-acyl serinol or, more specifically, N-oleoyl serinol.

In some aspects of the method, the composition is administered in a therapeutically effective amount, and/or the composition further comprises a pharmaceutically acceptable carrier, diluent, buffer, or excipient.

In some aspects of the method, the adenocarcinoma can be found in the digestive system of the subject. More specifically, the adenocarcinoma can be found in the liver, pancreas, small intestine, large intestine, colon, or stomach. In some aspects, the adenocarcinoma is hepatocellular carcinoma.

These and other advantages, aspects, and novel features of the present disclosure, as well as details of an illustrated embodiment thereof, will be more fully understood from the following description and drawings.

### BRIEF DESCRIPTION OF THE DRAWINGS

Various aspects of the present disclosure will now be described, by way of example only, with reference to the attached Figures, wherein:

FIG. 1A illustrates the experimental design for an animal model of NAFLD and hepatocellular carcinoma. The model induces all stages of fatty liver disease, with mice developing fatty liver and steatohepatitis by week 12, and progressing to cirrhosis and hepatocellular carcinoma by week 24.

FIG. 1B illustrates a first treatment group of FIG. 1A that receives N-acyl serinol (gavage of E. coli expressing pET28c:hm-N-acyl serinol synthase) at 12 weeks post-induction.

FIG. 1C illustrates a second treatment group of FIG. 1A that receives an N-acyl serinol point mutant (gavage of E. Coli expressing pET28c:hm-N-acyl serinol synthase with a point mutation) at 12-weeks post-induction.

FIG. 1D illustrates a control treatment group of FIG. 1B that receives no bacterial treatment 12 weeks post-induction.

FIG. 2A is a graph illustrating the weight gain of three treatment groups in an animal model of NAFLD and hepatocellular carcinoma.

FIG. 2B is a graph illustrating the food consumption of three treatment groups in an animal model of NAFLD and hepatocellular carcinoma.

FIG. 3A is a graph illustrating tumor number in livers isolated from three treatment groups at 24 weeks post-induction in an animal model of NAFLD and hepatocellular carcinoma.

FIGS. 3B and 3C are representative photographs of livers isolated from mice in an N-acyl serinol treatment group at 24 weeks post-induction in an animal model of NAFLD and hepatocellular carcinoma.

FIGS. 3D and 3E are representative photographs of livers isolated from mice in a point mutant treatment group at 24 weeks post-induction in an animal model of NAFLD and hepatocellular carcinoma.

FIG. 4A is a graph illustrating the liver weight of three treatment groups at 24 weeks post-induction in an animal model of NAFLD and hepatocellular carcinoma.

FIG. 4B is a graph illustrating the spleen weight of three treatment groups at 24 weeks post-induction in an animal model of NAFLD and hepatocellular carcinoma.

FIG. 5A is a graph illustrating liver steatohepatitis of three treatment groups at 24 weeks post-induction in an animal model of NAFLD and hepatocellular carcinoma.

FIG. 5B is a graph illustrating liver fat accumulation of three treatment groups at 24 weeks post-induction in an animal model of NAFLD and hepatocellular carcinoma.

FIG. 6A is a graph of collagen deposition (Sirius Red staining) in liver sections of three treatment groups at 24 weeks post-induction in an animal model of NAFLD and hepatocellular carcinoma.

FIG. 6B is a representative photograph of a liver section isolated from a control group at 24 weeks post-induction in an animal model of NAFLD and hepatocellular carcinoma.

FIG. 6C is a representative photograph of a liver section isolated from a point mutant treatment group at 24 weeks post-induction in an animal model of NAFLD and hepatocellular carcinoma.

FIG. 6D is a representative photograph of a liver section isolated from an N-acyl serinol treatment group at 24 weeks post-induction in an animal model of NAFLD and hepatocellular carcinoma.

FIG. 7 is a dose-response graph of tumor organoid treatment with various N-acyl amide compounds.

FIG. 8 is a dose response graph of tumor organoid treatment with small molecule cancer inhibitors.

FIG. 9A is a dose-response graph of human hepatocellular cancer and cholangiocarcinoma organoid treatment in patients with non alcoholic steatohepatitis with N-oleoyl glycine.

FIG. 9B is a dose-response graph of human hepatocellular cancer and cholangiocarcinoma organoid treatment in patients with non alcoholic steatohepatitis with N-oleoyl serinol.

### DETAILED DESCRIPTION

Unless otherwise defined, all technical and scientific terms used herein have the same meaning as commonly understood by one of ordinary skill in the art to which the methods described herein belong. The singular form “a”, “an” and “the” include plural referents unless the context clearly dictates otherwise. These articles refer to one or to more than one (i.e., to at least one).

The term “about” as used in connection with a numerical value throughout the specification and the claims denotes an interval of accuracy, familiar and acceptable to a person skilled in the art. In general, such interval of accuracy is +/−10%.

Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

The present disclosure relates to methods of treating a disease or disorder associated with abnormal G protein coupled receptor (GPCR) activity in a subject. In some aspects, the methods of treatment disclosed herein modulate (e.g., agonize or antagonize) GPCR activity in a subject to treat a disease or disorder. The term “abnormal” when used in the context of organisms, tissues, cells, or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics that are normal or expected for one cell or tissue type, might be abnormal for a different cell or tissue type.

Specifically described herein are methods of treating a disease or disorder in a subject, the method comprising administering a genetically engineered cell expressing a human microbial N-acyl synthase (hm-NAS) gene, an hm-NAS gene, and/or an N-acyl amide. More specifically, the methods described herein treat diseases or disorders in which GPCRs enriched in the gastrointestinal mucosa are dysregulated or have otherwise abnormal activity in a diseased or disordered state.

The N-acyl amides of the present disclosure are detailed in U.S. Publication No. 2020/0113950 entitled, “Human Microbiota Derived N-Acyl Amides for the Treatment of Human Disease”, which is incorporated by reference herein in its entirety. The genetically engineered cells expressing a human microbial N-acyl synthase (hm-NAS) gene and the hm-NAS genes of the present disclosure are also described in U.S. Publication No. 2020/0113950.

GPCRs of the present disclosure include, but are not limited to, ADCYAP1R1, ADORA3, ADRA1B, ADRA2A, ADRA2B, ADRA2C, ADRB1, ADRB2, AGTR1, AGTRL1, AVPR1A, AVPR1B, AVPR2, BAI1, BAI2, BAI3, BDKRB1, BDKRB2, BRS3, C3AR1, C5AR1, C5L2, CALCR, CALCRL-RAMP1, CALCRL-RAMP2, CALCRL-RAMP3, CALCR-RAMP2, CALCR-RAMP3, CCKAR, CCKBR, CCR1, CCR10, CCR2, CCR3, CCR4, CCR5, CCR6, CCR7, CCR8, CCR9, CCRL2, CHRM1, CHRM2, CHRM3, CHRM4, CHRM5, CMKLR1, CNR1, CNR2, CRHR1, CRHR2, CRTH2, CX3CR1, CXCR1, CXCR2, CXCR3, CXCR4, CXCR5, CXCR6, CXCR7, DARC, DRD1, DRD2L, DRD2S, DRD3, DRD4, DRD5, EBI2, EDG1, EDG3, EDG4, EDG5, EDG6, EDG7, EDNRA, EDNRB, F2R, F2RL1, F2RL3, FFAR1, FPR1, FPRL1, FSHR, G2A, GALR1, GALR2, GCGR, GHSR, GHSR1B, GIPR, GLP1R, GLP2R, GPR1, GPR101, GPR103, GPR107, GPR109A, GPR109B, GPR119, GPR12, GPR120, GPR123, GPR132, GPR135, GPR137, GPR139, GPR141, GPR142, GPR143, GPR146, GPR148, GPR149, GPR15, GPR150, GPR151, GPR152, GPR157, GPR161, GPR162, GPR17, GPR171, GPR173, GPR176, GPR18, GPR182, GPR20, GPR23, GPR25, GPR26, GPR27, GPR3, GPR30, GPR31, GPR32, GPR35, GPR37, GPR37L1, GPR39, GPR4, GPR45, GPR50, GPR52, GPR55, GPR6, GPR61, GPR65, GPR75, GPR78, GPR79, GPR83, GPR84, GPR85, GPR88, GPR91, GPR92, GPR97, GRPR, HCRTR1, HCRTR2, HRH1, HRH2, HRH3, HRH4, HTR1A, HTR1B, HTR1E, HTR1F, HTR2A, HTR2C, HTR5A, KISS1R, LGR4, LGR5, LGR6, LHCGR, LTB4R, MC1R, MC3R, MC4R, MC5R, MCHR1, MCHR2, MLNR, MRGPRD, MRGPRE, MRGPRF, MRGPRX1, MRGPRX2, MRGPRX4, MTNR1A, NMBR, NMU1R, NPBWR1, NPBWR2, NPFFR1, NPSR1B, NPY1R, NPY2R, NTSR1, OPN5, OPRD1, OPRK1, OPRL1, OPRM1, OXER1, OXGR1, OXTR, P2RY1, P2RY11, P2RY12, P2RY2, P2RY4, P2RY6, P2RY8, PPYR1, PRLHR, PROKR1, PROKR2, PTAFR, PTGER2, PTGER3, PTGER4, PTGFR, PTGIR, PTHR1, PTHR2, RXFP3, SCTR, SPR4, SSTR1, SSTR2, SSTR3, SSTR5, TAAR5, TACR1, TACR2, TACR3, TBXA2R, TRHR, TSHR(L), UTR2, VIPR1, and VIPR2.

In some aspects, the methods of treatment disclosed herein modulate the activity of GPR119 in the gastrointestinal (GI) tract. In some aspects, the disclosure provides methods of treating a disease in a subject, wherein the disease is associated with abnormal GPR119 activity and the method comprises administering a genetically engineered cell expressing a human microbial N-acyl synthase (hm-NAS) gene, an hm-NAS gene, and/or an N-acyl amide. More specifically, the N-acyl amide administered directly or the N-acyl amide resulting from administering a genetically engineered cell expressing an hm-NAS gene or an hm-NAS gene exhibit agonist activity for GPR119 in the GI tract. In some aspects, the N-acyl amide is N-acyl serinol or, more specifically, N-oleoyl serinol. In some aspects, the hm-NAS gene (including the hm-NAS gene expressed by the genetically engineered cell) is N-acyl serinol synthase.

A “disease”, as used herein, is a state of health of a subject wherein the subject cannot maintain homeostasis, and wherein if the disease is not ameliorated, the subject's health continues to deteriorate. In contrast, a “disorder” is a state of health in which the subject is able to maintain homeostasis, but in which the subject's state of health is less favorable than it would be in the absence of the disorder. Left untreated, a disorder does not necessarily cause a further decrease in the subject's state of health. A disease or disorder is “alleviated” if the severity of a sign or symptom of the disease or disorder, the frequency with which such a sign or symptom is experienced by a subject, or both, is reduced.

Diseases of the present disclosure include adenocarcinoma, and in particular, adenocarcinoma found in the digestive system of a subject. In some aspects, the methods described herein treat hepatocellular carcinoma in a subject.

As used herein, the terms “subject”, “individual”, and “patient” are interchangeable, and relate to vertebrates, preferably mammals. For example, mammals in the context of the disclosure are humans, non-human primates, domesticated animals such as dogs, cats, sheep, cattle, goats, pigs, horses, etc., laboratory animals such as mice, rats, rabbits, guinea pigs, etc., as well as animals in captivity such as animals in zoos. The term “animal” as used herein includes humans. The term “subject” may also include a patient, i.e., an animal, having a disease.

As used herein, “treat”, “treating”, or “treatment” refers to administering a genetically engineered cell expressing a human microbial N-acyl synthase (hm-NAS) gene, an hm-NAS gene, and/or an N-acyl amide, or compositions as described herein, to a subject in order to eliminate or reduce the clinical signs of the disease or disorder (i.e., adenocarcinoma) in the subject; arrest, inhibit, or slow the progression of the disease or disorder in the subject; and/or decrease the number, frequency, or severity of clinical symptoms and/or recurrence of disease or disorder in the subject who currently has or who previously had the disease or disorder. In particular, the term “treatment of a disease” includes curing, shortening the duration, ameliorating, slowing down, or inhibiting progression or worsening, or delaying the onset of clinical symptoms in a subject who has the disease or disorder.

As used herein, the terms “prophylactic”, “preventive”, “preventing”, and “prevention” relate to the prevention of the occurrence of a disease or disorder or the progression of a multi-stage disease or disorder from a less severe to a more severe stage.

In a first aspect, the disclosure provides a method of treating and/or preventing adenocarcinoma in a subject, the method comprising administering to the subject an N-acyl amide. “Adenocarcinoma” as used herein refers to a malignant neoplasm of epithelial cells, and, more specifically, a carcinoma derived from glandular tissue or in which the tumor cells form a glandular structure. Adenocarcinoma includes four subcategories: acinar, papillary, bronchoalveolar, and solid carcinoma with mucus formation. Exemplary adenocarcinomas include esophageal cancer, pancreatic cancer, prostate cancer, cervical cancer, stomach cancer, breast cancer, colon cancer, lung cancer, intestinal cancer, and liver cancer.

In a preferred aspect, the method comprises treating and/or preventing adenocarcinoma of the digestive system of a subject. “Digestive system” refers to the mouth, esophagus, stomach, small intestine, large intestine, colon, pancreas, liver, gallbladder, rectum, and anus. In some aspects, the adenocarcinoma is found in the liver, pancreas, small intestine, large intestine (including the colon), or stomach. The colon will be understood as being one segment of the large intestine, the others being the cecum, rectum, and anal canal.

In another preferred aspect, the method comprises treating and/or preventing hepatocellular carcinoma. “Hepatocellular carcinoma” will be understood to mean primary liver cancer (i.e., originating in hepatocytes), and is distinct from secondary liver cancer (i.e., a cancer that originates in another tissue and spreads to the liver). Diagnostic methods for hepatocellular carcinoma are known in the art, and include blood tests to measure liver function, imaging tests such as CT and MRI, and liver biopsy. Risk factors for hepatocellular carcinoma include hepatitis B or C, heavy and prolonged alcohol consumption, obesity, diabetes, and cirrhosis. Symptoms of hepatocellular carcinoma include nausea, loss of appetite, unintentional weight loss, fatigue, jaundice, swelling in the abdomen and/or legs, increased susceptibility to bleeding or bruising, and abdominal pain.

In some aspects, the method treats or prevents hepatocellular carcinoma resulting from end-stage liver disease, and, more specifically, from non-alcoholic fatty liver disease (NAFLD). NAFLD comprises multiple stages including simple fatty liver (steatosis), non-alcoholic steatohepatitis (NASH), fibrosis, and cirrhosis, which can result in liver cancer. A person at risk for developing heptatocellular carcinoma or end-stage liver disease would be a candidate for preventative therapy using the methods disclosed herein. In some aspects, a person at risk for developing hepatocellular carcinoma can exhibit signs of steatohepatitis, cirrhosis, or both.

In another aspect, the methods disclosed herein can prevent progression of liver disease (e.g. steatohepatitis, cirrhosis, and hepatocellular carcinoma) in subjects at risk for developing liver disease.

Clinical outcomes for measuring, analyzing, monitoring, or quantifying the effectiveness of the treatment and/or prevention methods as disclosed herein are known to one of ordinary skill and include but are not limited to, decreased liver inflammation in a subject as evidenced by decreased liver transaminases levels; decreased accumulation of liver fat in a subject as evidenced by decreased liver transaminases levels and/or imaging (e.g. ultrasound, MRI, CT scan); decreased liver fibrosis in a subject as evidenced by tissue biopsy and/or improvement in secondary measures of cirrhosis (e.g. portal hypertension, encephalopathy, and imaging (ultrasound/elastography, MRI, CT scan); decreased tumor number; and/or decreased tumor size.

The N-acyl amides of the present disclosure are detailed in U.S. Publication No. 2020/0113950, and are incorporated by reference. Exemplary N-acyl amides include those having Formula (1):

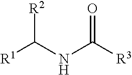

wherein R¹ is selected from the group consisting of carboxylate and CH₂OH; R² is selected from the group consisting of H, (C₃-C₄) alkyl-NH₃⁺, (C₃-C₄)alkyl-NH₂, C₂ alkyl-C(═O)NH₂, CH₂OH, and methyl; and R³ is selected from the group consisting of (C₉-C₁₈)alkyl, (C₉-C₁₈)alkenyl, wherein the (C₉-C₁₈)alkyl and (C₉-C₁₈)alkenyl are optionally substituted.

In some aspects, Formula (1) of the N-acyl amide is represented by one of Formulae (2)-(6):

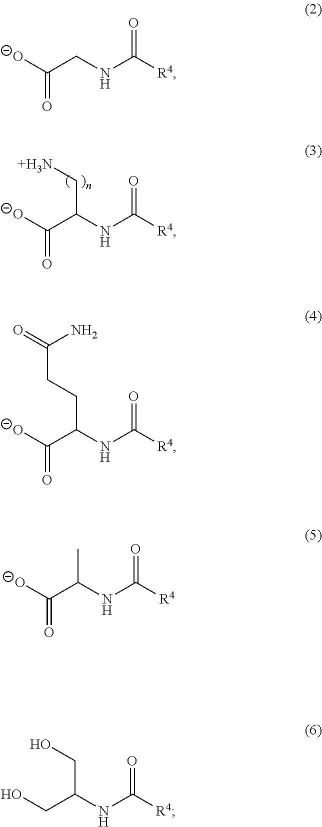

wherein R⁴ is selected from the group consisting of (C₉-C₁₈)alkyl, (C₉-C₁₈)alkenyl, wherein the (C₉-C₁₈)alkyl and (C₉-C₁₈)alkenyl are optionally substituted; and n is 3 or 4.

In some aspects of the method, Formulae (2)-(6) are represented by Formulae (7)-(11):

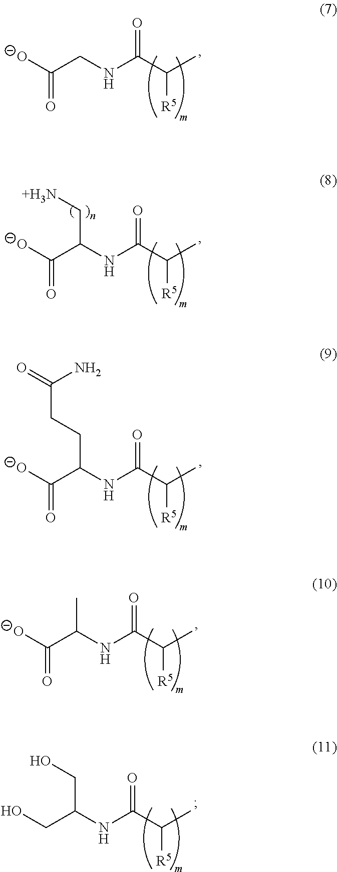

wherein R⁵ is independently selected from the group consisting of H and —OH; and m is an integer from 8 to 17.

In some aspects of the method, Formulae (2)-(6) are represented by Formulae (12)-(16):

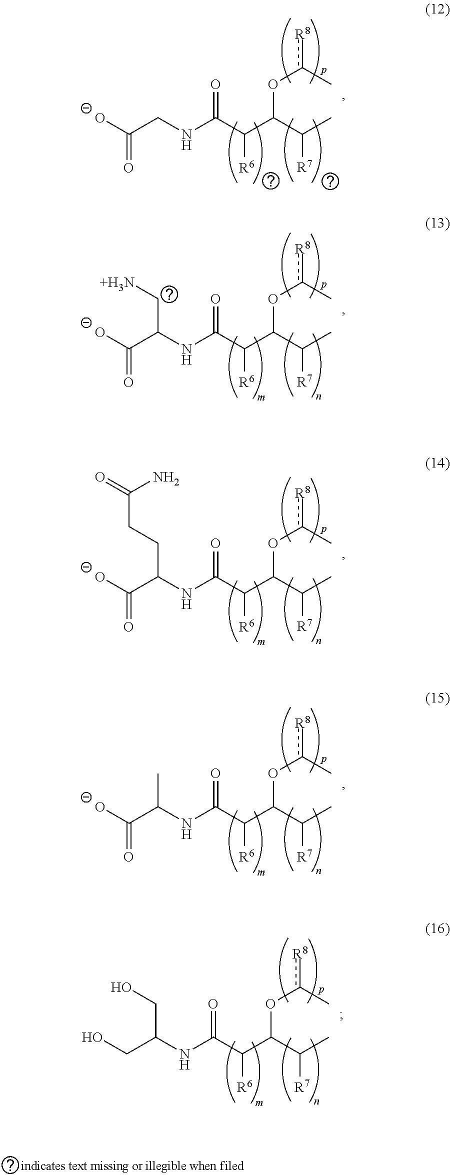

wherein R⁶, R⁷, and R⁸ are independently selected from the group consisting of H, —OH, and =0; m is an integer from 1 to 5; n is an integer from 2 to 15; p is an integer from 8 to 18; and q is an integer from 3 to 4.

In some aspects of the method, the N-acyl amide is selected from the group consisting of:

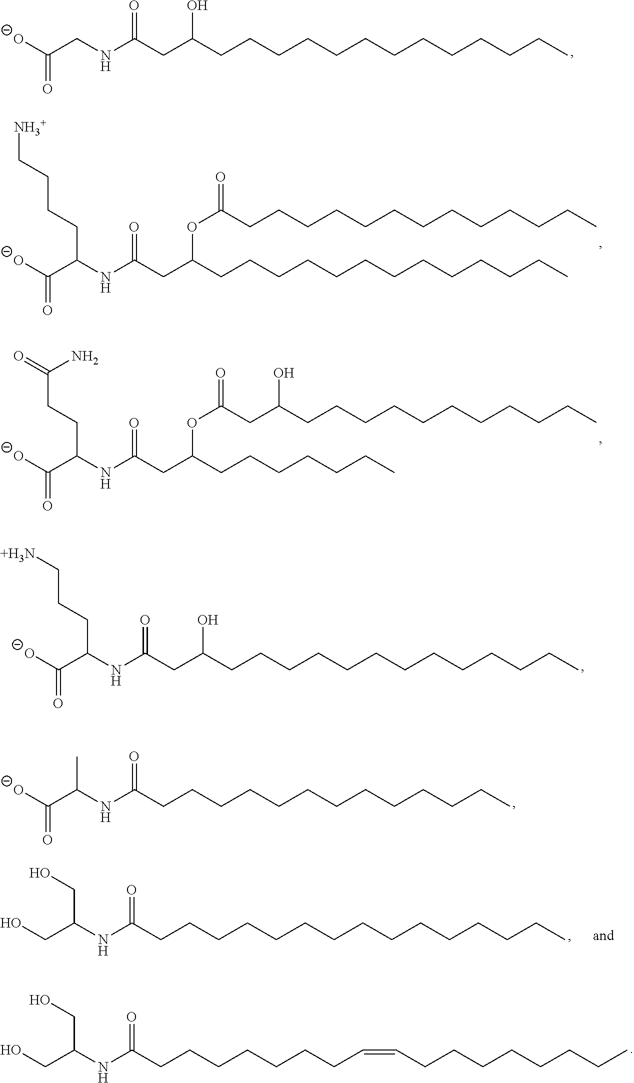

In some aspects of the method, the N-acyl amide is N-acyl serinol or, more specifically, N-oleoyl serinol. In some aspects, a method of treating adenocarcinoma in a subject comprises administering N-acyl serinol to the subject. In some aspects, a method of treating adenocarcinoma in a subject comprises administering N-oleoyl serinol to the subject.

In another aspect, the disclosure provides a method of treating and/or preventing adenocarcinoma in a subject, the method comprising administering to the subject a composition comprising a genetically engineered cell expressing a human microbial N-acyl synthase (hm-NAS) gene, an hm-NAS gene, and/or an N-acyl amide.

In some aspects of the method, the genetically engineered cell expressing a human microbial N-acyl synthase (hm-NAS) gene encodes an N-acyl synthase polypeptide that catalyzes synthesis of an N-acyl amide as described herein. Similarly, administering an hm-NAS gene to a subject results in the gene encoding an N-acyl synthase polypeptide that catalyzes synthesis of an N-acyl amide. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcnpt10n of a gene or cDNA, can be referred to as encoding the protein or other product of that gene or cDNA. The terms “expressing” or “expression” as used herein is defined as the transcription and/or translation of a particular nucleotide sequence driven by its promoter.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). The term “nucleic acid” typically refers to large polynucleotides.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein or peptide sequence. Polypeptides include any peptide or protein comprising two or more amino acids joined to each other by peptide bonds. As used herein, the term refers to both short chains, which also commonly are referred to in the art as peptides, oligopeptides, and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of poly-peptides, modified polypeptides, derivatives, analogs, fusion proteins, among others. The polypeptides include natural peptides, recombinant peptides, synthetic peptides, or a combination thereof.

In some aspects, the genetically engineered cell of the method is a non-pathogenic bacterial cell. “Non-pathogenic bacteria” refer to bacteria that are not capable of causing disease or harmful responses in a host. In some aspects, non-pathogenic bacteria are commensal bacteria. Examples of non-pathogenic bacteria include, but are not limited to, Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium in/antis, Bifidobacterium lactis, Bifidobacterium longum, Clostridium butyricum, Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii (Sonnenbom et al., 2009; Dinleyici et al., 2014; U.S. Pat. Nos. 6,835,376; 6,203,797; 5,589,168; 7,731,976). Endogenously pathogenic bacteria can be genetically engineered to provide reduced or eliminated pathogenicity. Non-pathogenic bacteria can be genetically engineered to enhance or improve desired biological properties (e.g., survivability). In a particular aspect, the non-pathogenic bacterial cell is E. coli.

Exemplary hm-NAS genes of the present disclosure are identified in Tables 1 and 2.

TABLE 1
Heterologously expressed hm-NAS genes

| Clone Number | EBIGene | Gene Size (bn) | Molecule Family |
|---|---|---|---|
| 1 | EFI7261 | 1191 | No production |
| 2 | EHB91285 | 921 | 1 |
| 3 | EEK17761 | 960 | No production |
| 5 | EEY82825 | 987 | 1 |
| 6 | EHP49568 | 969 | No production |
| 7 | EHG23013 | 1008 | 1 |
| 8 | EFA42931 | 999 | 1 |
| 9 | EFL47029 | 1005 | 1 |
| 10 | EHO75052 | 1005 | 1 |
| 11 | ADK95845 | 1011 | 1 |
| 12 | EFV04460 | 1017 | 1 |
| 13 | EHH01788 | 945 | 1 |
| 14 | EDY97076 | 1002 | 1 |
| 15 | CBW20928 | 1026 | 1 |
| 16 | EDS14876 | 1035 | 1 |
| 17 | EDO52243 | 990 | 1 |
| 18 | CBK67812 | 1029 | 1 |
| 19 | ACI09609 | 1713 | 3 |
| 21 | ABV66681 | 1716 | 2 |
| 24 | EHT12133 | 1731 | 2 |
| 26 | EFE54303 | 1743 | 2 |
| 27 | EFE94777 | 1734 | 2 |
| 29 | EER56350 | 768 | No production |
| 30 | EET45812 | 783 | 4 |
| 31 | ACS62992 | 846 | 4 |
| 33 | BAH33083 | 849 | No production |
| 35 | EFG73978 | 870 | No production |
| 36 | CAW29482 | 768 | 4 |
| 37 | EFH13337 | 813 | 4 |
| 38 | EGP09383 | 1041 | No production |
| 39 | EEV22085 | 1011 | No production |
| 40 | EEY94333 | 789 | No production |
| 41 | EFF83269 | 789 | No production |
| 42 | CAP01857 | 816 | 4 |
| 43 | EGP10046 | 804 | 5 |
| 50 | EFK33376 | 1854 | No production |
| 51 | EEK14630 | 1815 | No production |
| 52 | EFS97491 | 1848 | 2 |
| 53 | CBK85930 | 1713 | 2 |
| 54 | EHM48796 | 1713 | 2 |
| 55 | EEK89350 | 1596 | No production |
| 56 | EHL05550 | 1638 | 6 |
| 57 | EFV76279 | 1623 | 6 |
| 58 | GL883582 | 1576 | 6 |

Molecule Family 1 - N-acyl glycines
Molecule Family 2 - N-acylox-yacyl lysine
Molecule Family 3 - N-acyloxyacyl glutamine
Molecule Family 4 - N-acyl lysine/ornithine
Molecule Family 5 - N-acyl alanine
Molecule Family 6 -N-acyl serinols

TABLE 2
PFAM13444 related hm-NAS genes

| | N-acyl | | |
|---|---|---|---|
| | Amide | | |
| | Molecule | | Related Human Microbial Gene |
| PFAM13444 Gene EBI reference information | Family | E-Value | Identified in HMP reference genome |
| R6A3N1_9BACT/51-156 | 1 | 2.00E−22 | >ADDV01000044 Prevotella oris C735 |
| R6EH40_9BACT/51-155 | 1 | 3.00E−74 | >ADDV01000044 Prevotella oris C735 |
| R7PBT6_9BACT/52-156 | 1 | 6.00E−07 | >ADCT01000041 Prevotella sp. C561 |
| R7NN97_9BACE/51-155 | 1 | 0 | >AQHY01000032 Bacteroides massiliensis B84634 |
| A0A0C3RD59_9PORP/51-157 | 1 | 4.00E−13 | >GG705232 Bacteroides sp. 3 [symbol] 1 [symbol] 33FAA |
| A6L081_BACV8/51-155 | 1 | 0 | >ADKO01000098 Bacteroides vulgatus PC510 |
| A6LEV2_PARD8/51-155 | 1 | | >ACPW01000045 Parabacteroides sp. D13 |
| D4 [symbol] M [symbol] _9BACT/57-158 | 1 | | >ADKO01000098 Bacteroides vulgatus PC510 |
| D5EVS3_PRER2/52-157 | 1 | 2.00E−126 | >DS995534 Bacteroides dorei DSM 17855 |
| D6D060_9BACE/51-155 | 1 | 0 | >GG3705232 Bacteroides sp. 3 [symbol] 1 [symbol] 33FAA |
| E6SV [symbol] 0_BACT6/51-155 | 1 | 0 | >FP929032 Alistipes shahii WAL 8301 |
| CBK67812_CBK67812.1_Bacteroides_{—} | 1 | 0 | >GG703854 Prevotella copri DSM 18205 |
| xylanisolvensXB1A_hypothetical protein | | | |
| ENA_CBW20928_CBW20928.1_Bacteroides_{—} | 1 | 0 | >FP929033 Bacteroides xylanisolvens XB1A |
| fragilis_638R_putative_hemolysin_A | | | |
| ENA_EDO52243_EDO52243.1_Bacteroides_{—} | 1 | 0 | >GL882689 Bacteroides fluxus YIT 12057 |
| uniformis_ATCC_8492_hemolysin | | | |
| ENA_EDS14876_EDS14876.1_Bacteroides_{—} | 1 | 0 | >FP929033 Bacteroides xylanisolvens XB1A |
| stercoris_ATCC_43183_hemolysin | | | |
| ENA_EDY97076_EDY97076.1_Bacteroides_{—} | 1 | 0 | >JH636044 Bacteroides sp. 3_2_5 |
| plebeius_DSM_17135_hemolysin_{—} | | | |
| ENA_EEY82825_EEY82825.1_Bacteroides_{—} | 1 | 0 | >ACPT01000029 Bacteroides sp. D20 |
| sp._2_1_33B_hemolysin_{—} | | | |
| ENA_EFV04460_EFV04460.1_Prevotella_{—} | 1 | 0 | >ABFZ02000020 Bacteroides stercoris ATCC 43183 |
| salivae_DSM_15606_hemolysin [symbol] | | | |
| ENA_EHB91285_EHB91285.1_Alistipes_{—} | 1 | 0 | >ABQC02000004 Bacteroides plebeius DSM 17135 |
| indistinctus_YIT_12060_hypothetical_protein | | | |
| ENA_EHH01788_EHH01788.1_{—} | 1 | 0 | >GG705151 Bacteroides sp. 2_1_33B |
| Paraprevotella_clara_YIT_11840_hemolysin | | | |
| ENA_EHP49568_EHP49568.1_Odoribacter_{—} | 1 | 0 | >GL629647 Prevotella salivae DSM 15606 |
| lancus_YIT_12061_hypothetical_protein | | | |
| I3YLB0_ALIFI/56-157 | 1 | 0 | >JH370372 Alistipes indistinctus YIT 12060 |
| Q5L [symbol] _BACFN/51-155 | 1 | 0 | >JH376579 Paraprevotella clara YIT 11840 |
| Q8A247_BACTN/51-155 | 1 | 0 | >JH594596 Odoribacter laneus YIT 12061 |
| R5C642_9BACE/51-155 | 1 | 8.00E−120 | >FP929032 Alistipes shahii WAL 8301 |
| R5FQF1_9BACT/53- 157 | 1 | 1.00E−113 | >ACW101000002 Bacteroides sp. 2_1_56FAA |
| R5 [symbol] 942_9PORP/51-156 | 1 | 5.00E−22 | >JH636041 Bacteroides sp. 1_1_6 |
| R5JGR8_9BACE/51-155 | 1 | 0 | >KB905466 Bacteroides salyersiae WAL 10018 |
| R5KD71_9BACT/52-157 | 1 | 6.00E−171 | >GL629647 Prevotella salivae DSM 15606 |
| R5MMX8_9BACE/51-155 | 1 | 0 | >ACWH01000030 Bacteroides ovatus 3_8_47FAA |
| R5NZ [symbol] _9BACT/51-155 | 1 | 0 | >KB905466 Bacteroides salyersiae WAL 10018 |
| R5UEV5_9BACE/51-155 | 1 | 0 | >JH379426 Prevotella stercorea DSM 18206 |
| R5UP [symbol] 5_9PORP/51-157 | 1 | 0 | >ABJL02000006 Bacteroides intestinalis DSM 17393 |
| R5VW07_9BACE/51-155 | 1 | 0 | >JH376579 Paraprevotella clara YIT 11840 |
| R6B4U0_9BACT/52-156 | 1 | 0 | >AAVM02000009 Bacteroides caccae ATCC 43185 |
| R6BXV9_9BACT/52-157 | 1 | 0 | >GG703584 Prevotella copri DSM 18205 |
| R6DH [symbol] 5_9BACE/51-155 | 1 | 0 | >GG688329 Bacteroides finegoldii DSM 17565 |
| R6FKP [symbol] _9BACE/51-155 | 1 | 0 | >DS499674 Bacteroides stercoris ATCC 43183 |
| R6FUQ8_9BACT/52-158 | 1 | 0 | >JH379426 Prevotella stercorea DSM 18206 |
| R6KTM3_9BACE/51-155 | 1 | 0 | >ACCH01000127 Bacteroides cellulosilyticus DSM 14838 |
| R6LN [symbol] 9_9BACE/51-154 | 1 | 0 | >AFBM01000001 Bacteroides clarus YIT 12056 |
| R6MX [symbol] 6_9BACE/51-155 | 1 | 0 | >DS981492 Bacteroides coprocola DSM 17136 |
| R6QE29_9BACT/52-157 | 1 | 0 | >GG703854 Prevotella copri DSM 18205 |
| R6S950_9BACE/51-155 | 1 | 0 | >GG688329 Bacteroides finegoldii DSM 17565 |
| R6SC6 [symbol] _9BACE/51-155 | 1 | 0 | >ACBW01000097 Bacteroides coprophilus DSM 18228 |
| R6VUA [symbol] _9BACT/56-157 | 1 | 0 | >FP929032 Alistipes shahii WAL 8301 |
| R6XGV7_9BACT/52-157 | 1 | 6.00E−106 | >GG703854 Prevotella copri DSM 18205 |
| R6Y [symbol] B5_9BACE/51-155 | 1 | 2.00E−121 | >ACTC01000036 Bacteroides sp. 4_1_36 |
| R7DDR3_9PORP/51-155 | 1 | 0 | >ACWX01000035 Tannerella sp. 6 [symbol] 1 [symbol] 58FAA CT [symbol] |
| R7E [symbol] P8_9BACE/51-155 | 1 | 0 | >ACPT01000029 Bacteroides sp. D20 |
| R7F021_9BACT/51-157 | 1 | 2.00E−11 | >AFZZ01000132 Prevotella stercorea DSM 18206 |
| R7HSG0_9BACT/37-143 | 1 | 2.00E−26 | >AFZZ01000132 Prevotella stercorea DSM 18206 |
| R [symbol] YP9_9BACT/59-165 | 1 | 1.00E−58 | >JH379426 Prevotella stercorea DSM 18206 |
| R7JHM4_9BACT/51-152 | 1 | 0 | >ABFK02000017 Alistipes putredinis DSM 17216 |
| E6K481_9BACT/52-156 | 1 | 0 | >AEPD01000010 Prevotella buccae ATCC 33574 |
| ENA_ADK95845_ADK95845.1_Prevotella_{—} | 1 | 0 | >CP002122 Prevotella melaninogenica ATCC 25845 |
| melaninogenica_ATCC_25845_hemolysin | | | |
| ENA_EF [symbol] 7261_EF [symbol] 7261.1_{—} | 1 | 0 | >ADCM01000011 Bacteroidetes oral taxon 274 str. F0058 |
| Bacteroidetes_oral_taxon_274_{—} | | | |
| str._F0058_hemolysin | | | |
| ENA_EHG23013_EHG23013.1_{—} | 1 | 0 | >JH376829 Prevotella sp. oral taxon 302 str. F0323 |
| Alloprevotella_rava_F0323_{—} | | | |
| hypothetical_protein | | | |
| ENA_EHO75052_EHO75052.1_Prevotella_{—} | 1 | 0 | >JH594521 Prevotella micans F0438 |
| micans_F0438_hypothetical_protein | | | |
| F2KX [symbol] 9_PREDF/64-168 | 1 | 0 | >CP002589 Prevotella denticola F0289 |
| F9D3S1_PREDD/52-156_1 | 1 | 0 | >GL982488 Prevotella dentalis DSM 3688 |
| [symbol] YUM9_PRE [symbol] 7/53-157 | 1 | 1.00E−98 | >GG703886 Prevotella oris F0302 |
| Q7MTR9_PORG [symbol] /53-158 | 1 | 0 | >AJZS01000078 Porphyromonas gingivalis W50 |
| R5CSR0_9BACT/52-157 | 1 | 3.00E−115 | >AWEY01000007 Prevotella baroniae F0067 |
| R5GFN8_9BACT/51-155 | 1 | 4.00E−29 | >ACZS01000081 Prevotella sp. oral taxon 472 str. F0295 |
| R5Q4D6_9BACT/52-157 | 1 | 6.00E−107 | >AWET01000051 Prevotella pleuritidis F0068 |
| R6W2Q2_9BACT/52-156 | 1 | 3.00E−160 | >GL872283 Prevotella multiformis DSM 16608 |
| R7CYB8_9BACE/51-155 | 1 | 3.00E−15 | >CP002122 Prevotella melaminogenica ATCC 25845 |
| W0EP20_9PORP/51-155 | 1 | 5.00E−43 | >AWEY01000007 Prevotella baroniae F0067 |
| C7M608_CAPOD/352-453 | 2 | 0 | >AMEV01000023 Capnocytophaga sp. oral taxon 324 str. F0483 |
| ENA_EEK14630_EEK14630.1_Capnocytophaga_{—} | 2 | 0 | >ACLQ01000018 Capnocytophaga gingivalis ATCC 33624 |
| gingivalis_ATCC_33624_Acyltransferase | | | |
| ENA_EFS97491_EFS97491.1_{—} | 2 | 0 | >AKFV01000035 Capnocytophaga ochracea str. Ho [symbol] 25 |
| Capnocytophaga_ochracea_F0287_{—} | | | |
| Acyltransferase | | | |
| F9YU78_CAPCC/351-452 | 2 | 8.00E−173 | >AMEV01000023 Capnocytophaga sp. oral taxon 324 str. F0483 |
| H1Z9S5_MYROD/346-447 | 2 | 2.00E−40 | >ALNN01000028 Capnocytophaga sp. CM59 |
| ENA_EFA42931_EFA42931.1_Prevotella_{—} | 1 | 0 | >GG704783 Prevotella bergensis DSM 17361 |
| bergensis_DSM_17361_hemolysin | | | |
| A0A095ZG93_9BACT/52-156 | 1 | 0 | >ADEG01000046 Prevotella buccalis ATCC 35310 |
| E7RNE3_9BACT/52-156 | 1 | 0 | >AEPE02000002 Prevotella oralis ATCC 33269 |
| ENA_EEK17761_EEK17761.1_{—} | 1 | 0 | >ACLR01000009 Porphyromonas uenonis 60-3 |
| Porphyromonas_uenonis_60-3_{—} | | | |
| hemolysin | | | |
| ENA_EFL47029_EFL47029.1_Prevotella_{—} | 1 | 0 | >AEDO01000009 Prevotella disiens FB035-09AN |
| disiens_FB035-09AN_hemolysin | | | |
| F4KL89_PORAD/55-160 | 1 | 0 | >AENO01000054 Porphyromonas asaccharolytica PR426713P- [symbol] |
| I4Z8L9_9BACT/52-156 | 1 | 0 | >ADFO01000053 Prevotella bivia JCV [symbol] MP010 |
| R6CE12_9BACE/51-155 | 1 | 1.00E−11 | >AEDO01000009 Prevotella disiens FB035-09AN |
| R6XAK6_9BACT/52-156 | 1 | 1.00E−120 | >AEPE02000002 Prevotella oralis ATCC 33269 |
| ENA_EHL05550_EHL05550.1_{—} | 6 | 0 | >JH414482 Desulfitobacterium hafniense DP7 |
| Desulfitobacterium_hafniense_{—} | | | |
| DP7_aminotransferase_class_V_{—} | | | |
| ENA_EFV76279_EFV76279.1_Bacillus_{—} | 6 | 0 | >GL635754 Bacillus sp. 2_A_57_CT2 |
| sp._2_A_57_CT2_serine-pyruvate [symbol] | | | |
| aminotransferase | | | |
| A6T596_KLEP7/322-423 | 2 | 0 | >JH930419 Klebsiella pneumoniae subsp. pneumoniae WGLW2 |
| D8MWX6_ERWBE/367-468 | 2 | 3.00E−147 | >GG753567 Serratia odorifera DSM 4582 |
| ENA_EFE94777_EFE94777.1_Serratia_{—} | 2 | 0 | >GG753567 Serratia odorifera DSM 4582 |
| odorifera_DSM [symbol] 4582_Acyltransferase | | | |
| Q6CZN2_PECAS/322-423 | 2 | 2.00E−109 | >ADBY01000051 Serratia odorifera DSM 4582 |
| A0A0B5CH45_NE [symbol] EG/32-132 | 4 | 0 | >ADBF01000232 Neisseria elongata subsp. |
| | | | glycolytica ATCC 29315 |
| E5UJR0_NEIMU/32-132 | 4 | 0 | >ACRG01000005 Neisseria mucosa C102 |
| ENA_EET45812_EET45812.1_Neisseria_{—} | 3 | 0 | >ACKO02000002 Neisseria sicca ATCC 29256 |
| sicca_ATCC_29256_hypothetical_protein | | | |
| ENA_ACI09609_ACI09609.1_Klebsiella_{—} | 3 | 0 | >ACXA01000063 Klebsiella sp. 1_1_55 |
| pneumoniae_342_conserved_hypothetical [symbol] | | | |
| protein | | | |
| A4W746_ENT38/322-423 | 2 | 0 | >FP929040 Enterobacter cloacae |
| | | | supsp. cloacae NCTC 9394 |
| ENA_CBK85930_CBK85930.1_Enterobacter_{—} | 2 | 0 | >FP929040 Enterobacter cloacae |
| cloacae_subsp._cloacae_NCTC_9394 [symbol] | | | supsp. cloacae NCTC 9394 |
| Putative [symbol] hemolysin [symbol] | | | |
| ENA_EFE54303_EFE54303.1_Providencia_{—} | 2 | 0 | >ACC [symbol] 02000039 Providencia rettgeri DSM 1131 |
| rettgeri_DSM_1131_Acyltransferase | | | |
| ENA_EHM48796_EHM48796.1_{—} | 2 | 0 | >JH417874 Yokenella regensburgei ATCC 43003 |
| Yokenella_regensburgei_ATCC_{—} | | | |
| 43003_Acyltransferase | | | |
| F9ZAJ4_ODOSD/341-443 | 2 | 0 | >JH594597 Odoribacter lancus YIT 12061 |
| G9Z3T1_9ENTR/322-423 | 2 | 0 | >JH417874 Yokenella regensburgei ATCC 43003 |
| R5UYM1_9PORP/338-439 | 2 | 0 | >ADMC01000028 Odoribacter lancus YIT 12061 |
| ENA_ACS62992_ACS62992.1_{—} | 4 | 0 | >GL520222 Ralstonia sp. 5_7_47FAA |
| Ralstonia_pickettii_12D_{—} | | | |
| conserved_hypothetical_{—} | | | |
| protein [symbol] | | | |
| ENA_CAW29482_CAW29482.1_{—} | 4 | 0 | >ACW [symbol] 01000206 Pseudomonas sp. 2_1_26 |
| Pseudomonas_aeruginosa_{—} | | | |
| LESB58_putative_hemolysin [symbol] | | | |
| A0A089UDH2_9ENTR/323-424 | 2 | 0 | >ALNJ01000086 Klebsiella sp. OBRC7 |
| E6WAC8_PANSA/322-423 | 2 | 7.00E−59 | >GL892086 Enterobacter hormaechei ATCC 49162 |
| ENA_EHT12133_EHT12133.1_{—} | 2 | 0 | >ALNJ01000086 Klebsiella sp. OBRC7 |
| Raoultella_ornithinolytica_{—} | | | |
| 10-5246 [symbol] hypothetical [symbol] | | | |
| protein | | | |
| G7LV45_9ENTR/322-423 | 2 | 5.00E−105 | >ALNJ01000086 Klebsiella sp. OBRC7 |
| ENA_EER56350_EER56350.1_{—} | 4 | 0 | >ACQV01000022 Neisseria flavescens SK114 |
| Neisseria_flavescens_SK114 [symbol] | | | |
| hypothetical [symbol] protein [symbol] | | | |
| A0A077KL [symbol] 9_9FLAO/353-454 | 2 | 0 | >GL379781 Chryseobacterium gleum ATCC 35910 |
| A7MLT3_CROS8/322-423 | 2 | 1.00E−178 | >AMLL01000012 Klebsiella pneumoniae subsp. |
| | | | pneumoniae WGLW1 |
| ENA_EFK33376_EFK33376.1_{—} | 2 | 0 | >GL379781 Chryseobacterium gleum ATCC 35910 |
| Chryseobacterium_gleum_ATCC_{—} | | | |
| 35910_Acyltransferase | | | |
| ENA_CAP01857_CAP01857.2_{—} | 4 | 0 | >ACQB01000026 Acinetobacter baumannii ATCC 19606 |
| Acinetobacter_baumannii_SDF_{—} | | | |
| conserved_hypothetical_protein | | | |

[symbol]

indicates data missing or illegible when filed

In some aspects, a method of treating adenocarcinoma in a subject comprises administering a composition comprising a genetically engineered cell expressing the N-acyl serinol synthase gene, the N-acyl serinol synthase gene, or N-acyl serinol to the subject. In some aspects, the N-acyl serinol is N-oleoyl serinol.

In another aspect, the disclosure provides a method of treating and/or preventing liver cancer in a subject, the method comprising administering to the subject a composition comprising a genetically engineered cell expressing an hm-NAS gene, an hm-NAS gene, and/or an N-acyl amide. “Liver cancer” will be understood to include primary and metastatic liver cancer. More specifically, “liver cancer” refers to hepatocellular carcinoma, cholangiocarcinoma (bile duct cancer), and metastatic liver cancer.

In some aspects, a method of treating liver cancer in a subject comprises administering a composition comprising a genetically engineered cell expressing the N-acyl serinol synthase gene, the N-acyl serinol synthase gene, or N-acyl serinol to the subject. In some aspects, the N-acyl serinol is N-oleoyl serinol.

In a further aspect, the disclosure provides a method of treating and/or preventing adenocarcinoma in a subject using a live biotherapeutic, the method comprising administering a genetically engineered cell expressing a human microbial N-acyl synthase (hm-NAS) gene, wherein the hm-NAS gene encodes an N-acyl synthase polypeptide. As used herein, a “biotherapeutic” refers to a compound or substance produced from biological sources, such as a living organism, rather than chemical synthesis. “Live biotherapeutic” refers to a living organism that when administered to a subject confers a health benefit to the subject. More specifically, “live biotherapeutic” as used herein refers to a living organism that when administered to a subject is applicable to the prevention, treatment, and/or cure of a disorder and/or disease. The live biotherapeutic disclosed herein (i.e., the genetically engineered cell expressing an hm-NAS gene) synthesizes N-acyl amide within the cell and releases it into the subject following administration. In some aspects, the N-acyl amide is synthesized by the genetically engineered cells and then into the gastrointestinal tract of the subject.

In some aspects, a method of treating adenocarcinoma in a subject comprises administering a live biotherapeutic to the subject, wherein the live biotherapeutic is a composition comprising a genetically engineered cell expressing the N-acyl serinol synthase gene.

In certain aspects, the disclosure provides methods of treatment and/or prevention comprising administration of a composition (e.g., a pharmaceutical composition). Such compositions comprise a genetically engineered cell expressing an hm-NAS gene, an hm-NAS gene, an N-acyl amide, or a combination thereof. When administered to a subject, the genetically engineered cell expressing an hm-NAS gene encodes an N-acyl synthase polypeptide that catalyzes synthesis of an N-acyl amide. Similarly, when an hm-NAS gene is administered to a subject, the hm-NAS gene encodes an N-acyl synthase polypeptide that catalyzes synthesis of an N-acyl amide.

In certain aspects, the above compositions can be formulated with a pharmaceutically acceptable carrier, excipient, diluent, buffer, or stabilizer. In certain aspects, such compositions are suitable for administration to a human, or a non-human mammal or animal, via any one or more route of administration using methods known in the art. The route and/or mode of administration will vary depending upon the desired results. The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also contain compatible solid or liquid fillers, diluents or encapsulating substances, which are suitable for administration to a human. Other contemplated carriers, excipients, and/or additives, which may be utilized in the formulations described herein include, for example, flavoring agents, antimicrobial agents, sweeteners, antioxidants, antistatic agents, lipids, protein excipients such as serum albumin, gelatin, casein, salt-forming counter-ions such as sodium and the like. These and additional known pharmaceutical carriers, excipients and/or additives suitable for use in the formulations described herein are known in the art, e.g., as listed in “Remington: The Science & Practice of Pharmacy”, 21st ed., Lippincott Williams & Wilkins, (2005), and in the “Physician's Desk Reference”, 60th ed., Medical Economics, Montvale, N.J. (2005). Pharmaceutically acceptable carriers can be selected that are suitable for the mode of administration, solubility and/or stability desired or required.

The compositions or therapeutic compositions described herein comprise active agents (i.e., a genetically engineered cell expressing an hm-NAS gene; an hm-NAS gene, an N-acyl amide; or a combination thereof) in a concentration resulting in a w/v appropriate for a desired dose. In certain aspects, the active agent is present in an “effective amount” or a “therapeutically effective amount”. The terms “effective amount” or “therapeutically effective amount” as used herein, refers to an amount or dosage level of an active ingredient that is effective in achieving a desired therapeutic response (e.g., treating adenocarcinoma) for a particular subject, composition, and mode of administration, without being toxic to the subject. The amount will depend upon a variety of pharmacokinetic factors including the activity of the particular compositions employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

Therapeutic compositions can be formulated for particular routes of administration, such as oral, nasal, pulmonary, topical (including buccal and sublingual), rectal, vaginal and/or parenteral administration. The phrases “parenteral administration” and “administered parenterally” as used herein refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The formulations (i.e., active and inactive agents) may be in unit dosage form and may be prepared by any known method. Actual dosage levels of the active ingredients in the compositions may be varied to obtain an amount of the active ingredient that is effective to achieve the desired therapeutic response for a particular patient, composition, and mode of administration, without being toxic to the patient (e.g., “a therapeutically effective amount”).

### Examples

### Example 1: Animal Model of Hepatocellular Carcinoma

To induce Nonalcoholic Fatty Liver Disease (NAFLD)/Nonalcoholic Steatohepatitis (NASH), 4-week-old C57BL/6 mice (Jackson Labs) were fed a high fat, high fructose western diet (TD.120528, Harlan Teklad) and high fructose, high glucose water (23.1 g/L d-fructose+18.9 g/L d-glucose). Mice were also given an intraperitoneal injection of CCl4 (0.2 μl/gm of body weight) once per week to induce acute liver injury mediated by reactive oxygen species. In this validated model, mice progress through all stages of fatty liver disease, developing fatty liver, steatohepatitis, and early bridging fibrosis by week 12, and progressing to cirrhosis and hepatocellular carcinoma by week 24 (T. Tsuchida et al, A simple diet- and chemical-induced murine NASH model with rapid progression of steatohepatitis, fibrosis and liver cancer. Journal of Hepatology 69, 385-395 (2018)). After 12 weeks, mice were randomly assigned to one of three treatment groups: Group I: N-acyl serinol treatment (bacterial gavage with E. coli expressing pET28c:hm-N-acyl serinol synthase), Group II: mutant N-acyl serinol treatment (bacterial gavage with E. Coli expressing pET28c:hm-N-acyl serinol synthase mutant), or Group III: control (no bacterial gavage). E. coli expressing pET28c:hm-N-acyl serinol synthase express N-acyl serinol synthase, which catalyzes synthesis of N-acyl serinol. There are 5 N-acyl serinol metabolites, the majority of which are N-oleoyl serinol and N-palmitoyl serinol. In the present disclosure, the major N-acyl serinol of interest is N-oleoyl serinol. Once synthesized, the N-acyl serinol is secreted from the E. coli into the gastrointestinal tract (GI) of the mouse. E. Coli expressing pET28c:hm-N-acyl serinol synthase mutant (i.e. “point mutants”) have a single base pair mutation rendering the N-acyl synthase ineffective. Thus, N-acyl serinol is not synthesized in the bacterial cell and released into the GI tract of the mouse. (L. J. Cohen et al., Commensal bacteria make GPCR ligands that mimic human signalling molecules. Nature 549, 48-53 (2017)).

After bacterial gavage of Groups 1 and 2, all groups were supplemented with kanamycin 35 μg ml-1 and 25 mM isopropyl β-d-1-thiogalactopyranoside IPTG in their drinking water. Mice were gavaged twice weekly (10⁸ CFU/gavage) from weeks 12-24. At week 24, mice were sacrificed. At the time of sacrifice, Group 1 had 14 mice (n=14), Group 2 had 9 mice (n=9), and Group 3 had 15 mice (n=15).

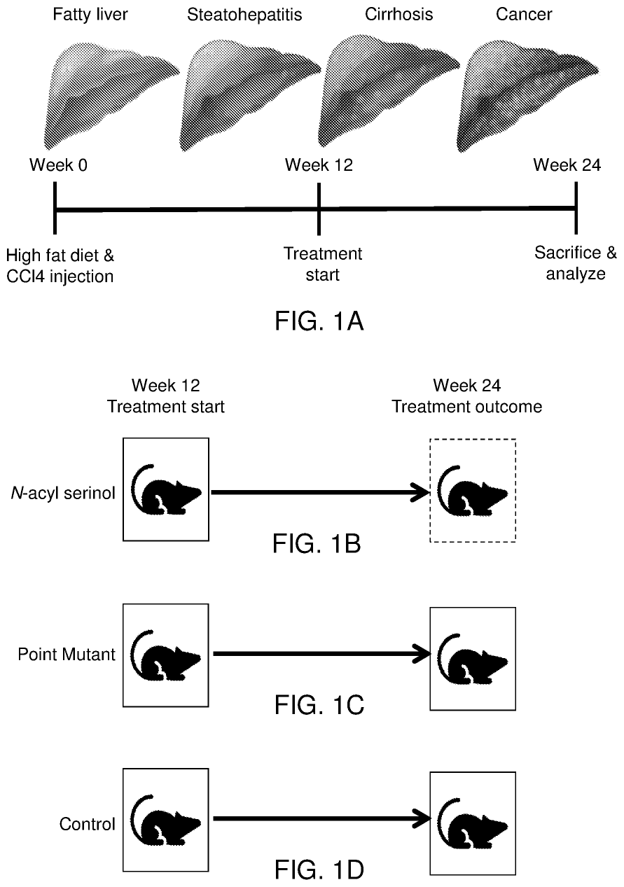

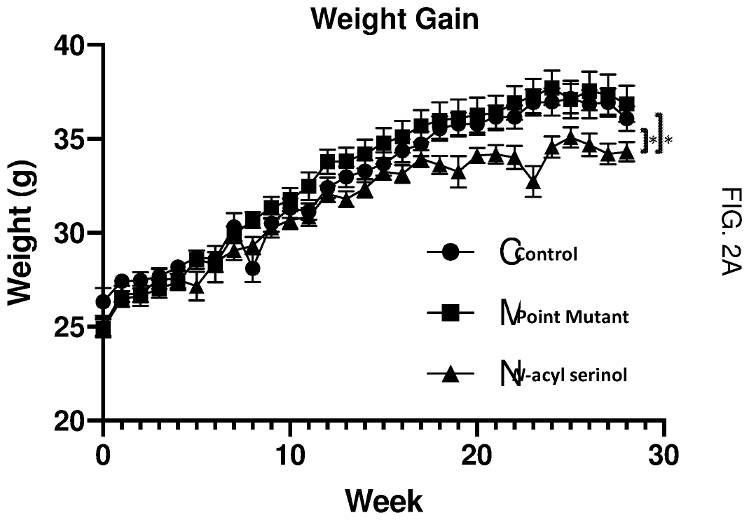

The experimental setup of Example 1 is shown in FIGS. 1A-1D. As illustrated in FIG. 1A, the model induces fatty liver and steatohepatitis from weeks 0-12 and cirrhosis and hepatocellular carcinoma from weeks 12-24.

### Example 2: Weight Gain and Food Consumption

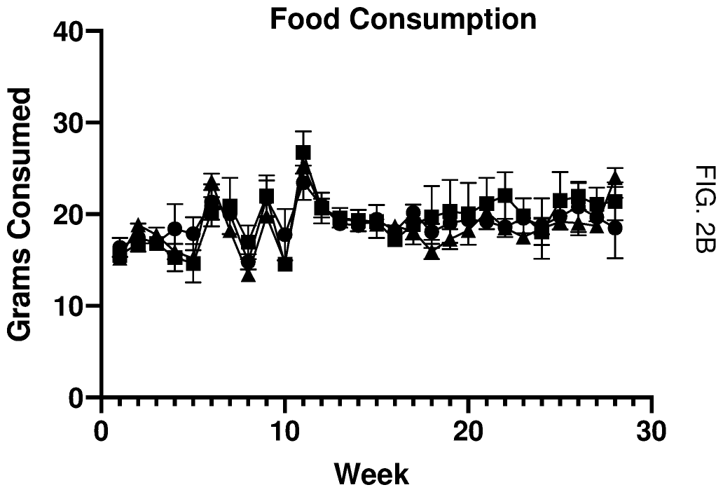

Mice were weighed and their food consumption measured weekly for 29 weeks. These results are shown in FIGS. 2A and 2B. There was a statistically significant decrease in weight gain for N-acyl serinol treatment mice (Group I) as compared to point mutant mice (Group II) from weeks 13-29, and a statistically significant decrease in weight gain for N-acyl serinol treatment mice (Group I) as compared to control mice (Group III) from weeks 19-29. There was no significant difference in weight gain between point mutant mice (Group II) and control mice (Group III) at any time point. There was no significant difference in food consumption between Groups I-III. Mice were compared by multiple t-tests with FDR correction for multiple tests at each week. Graphs mean+/−s.e.m. *p<0.05. n=14 (Group I); n=9 (Group II); n=15 (Group III).

These data demonstrate mice receiving N-acyl serinol treatment exhibit less weight gain compared to mice that do not receive N-acyl serinol treatment, which suggests the activity of N-acyl serinol in the GI tract reduces fat accumulation and/or increases energy expenditure.

### Example 3: Liver Tumor Count

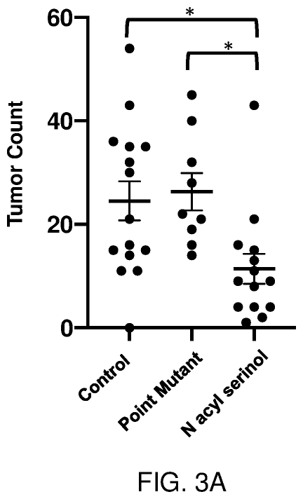

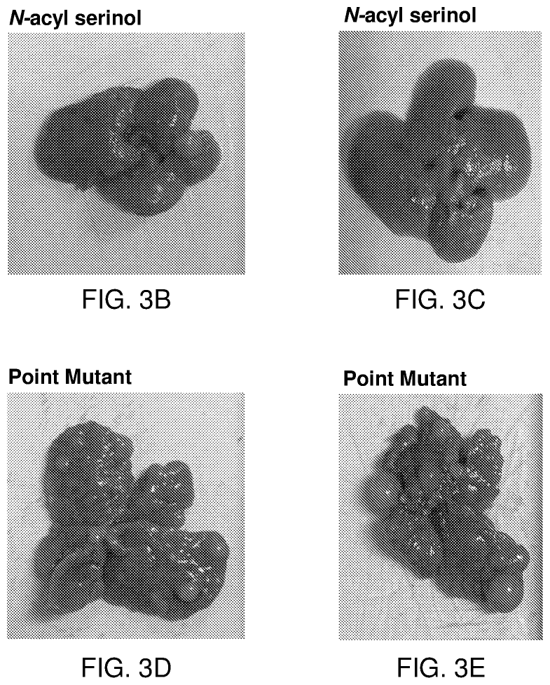

Livers were isolated from Groups I-III at the time of sacrifice (i.e., 24 weeks post-induction), and liver tumors were counted immediately thereafter. As shown in FIG. 3A, there was a statistically significant decrease in tumor number for N-acyl serinol treatment mice (Group I) as compared to point mutant mice (Group II) and control mice (Group III) at 24 weeks. There was no significant difference in tumor number between point mutant mice (Group II) and control mice (Group III). Graph mean+/−s.e.m. *p<0.05. n=14 (Group I); n=9 (Group II); n=15 (Group III).

FIGS. 3B and 3C are representative photographs of livers isolated from the N-acyl serinol treatment group at 24 weeks. FIGS. 3D and 3E are representative photographs of livers isolated from the point mutant treatment group at 24 weeks. The difference in tumor accumulation can be readily appreciated in these images.

These data demonstrate mice receiving N-acyl serinol treatment exhibit a lower tumor count as compared to point mutant and control mice. This suggests N-acyl serinol treatment reduces tumor formation in NAFLD and may prevent, reverse, or slow the progression of end-stage hepatocellular carcinoma.

### Example 4: Liver and Spleen Weight

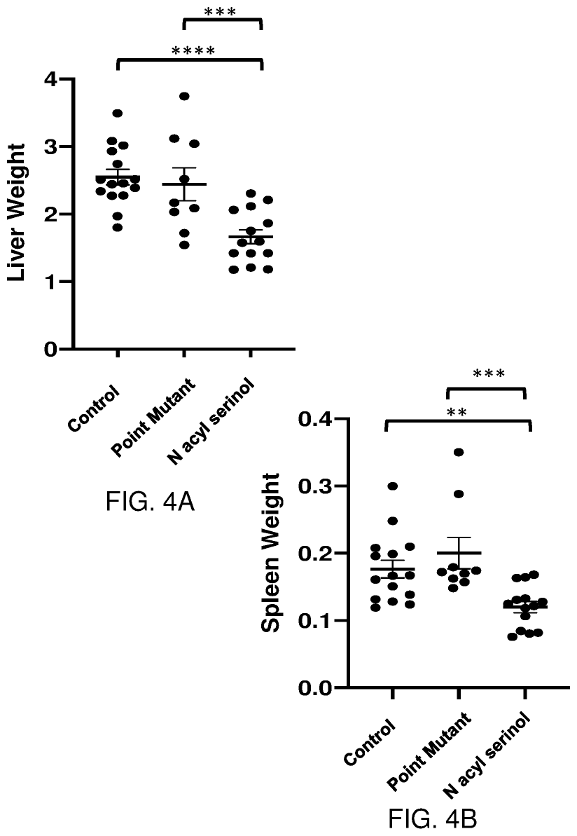

Spleens and livers were weighed from each animal of Groups I-III at the time of sacrifice (i.e., 24 weeks post-induction). As illustrated in FIGS. 4A and 4B, there was a statistically significant decrease in both liver (FIG. 4A) and spleen (FIG. 4B) weight for N-acyl serinol treatment mice (Group I) as compared to point mutant mice (Group II) and control mice (Group III) at 24 weeks. There was no significant difference in tumor number between point mutant mice (Group II) and control mice (Group III). Mice were compared by one-way ANOVA with Tukey's test for multiple comparisons. Graphs mean+/−s.e.m. *p<0.05 **p<0.01 ***p<0.001 ****p<0.0001. n=14 (Group I); n=9 (Group II); n=15 (Group III).

As increased liver and spleen weight positively correlates with liver disease severity, these data suggest N-acyl serinol treatment lessens disease severity in NAFLD and/or cirrhosis. Further, these data suggest N-acyl serinol treatment prevents the onset of liver cancer (e.g. hepatocellular carcinoma). These data also suggest N-acyl serinol treatment can lessen the clinical severity of liver disease symptoms such as cirrhosis.

### Example 5: Liver Histology and NASH Clinical Research Network (CRN) Score

After measuring weight and tumor number, left liver lobes were fixed in 10% formalin, paraffin embedded, microtome sectioned, and stained with H&E and Sirius Red. NASH activity was evaluated by a blinded pathologist who assessed NAFLD activity score (NAS) including lobular inflammation, steatosis, hepatocyte ballooning, and fibrosis stage.

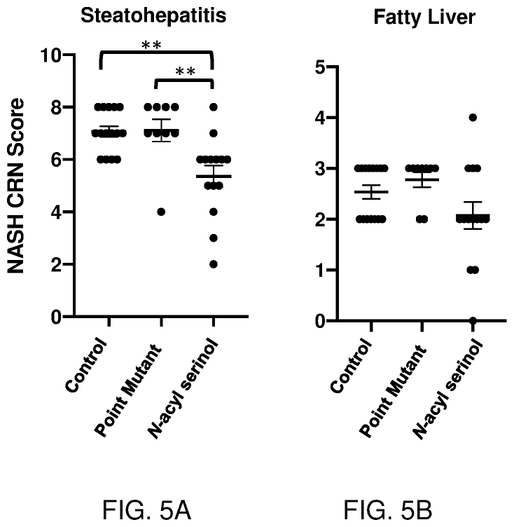

Liver steatohepatitis and fatty liver of Groups I-III, as measured by NASH Clinical Research Network (CRN) score, are shown in FIGS. 5A and 5B, respectively. There was a statistically significant decrease in steatohepatitis for N-acyl serinol treatment mice (Group I) as compared to point mutant (Group II) and control (Group III) mice. There was no significant difference between point mutant mice (Group II) and control mice (Group III). N-acyl serinol treatment mice (Group I) showed a non-statistically significant decrease in accumulated liver fat as compared to point mutant (Group II) and control (Group III) mice. Mice were compared by one-way ANOVA with Tukey's test for multiple comparisons. Graphs mean+/−s.e.m. **p<0.01. n=14 (Group I); n=9 (Group II); n=15 (Group III).

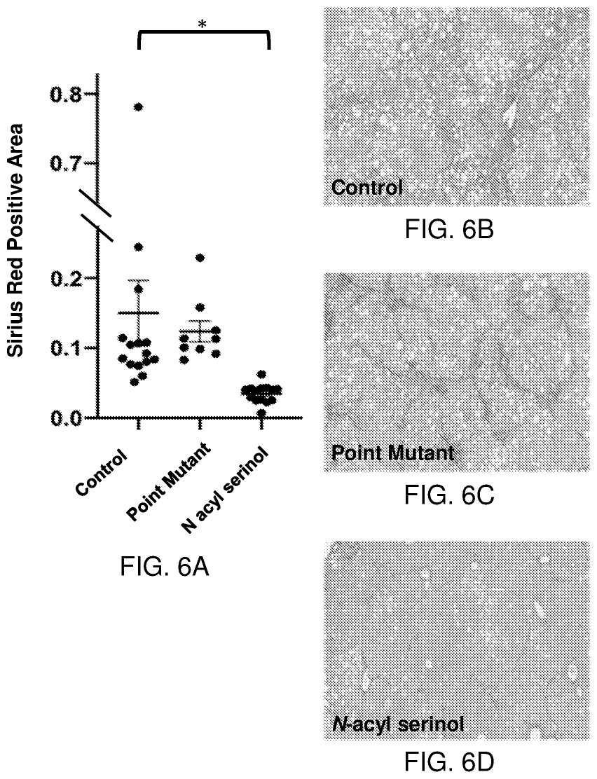

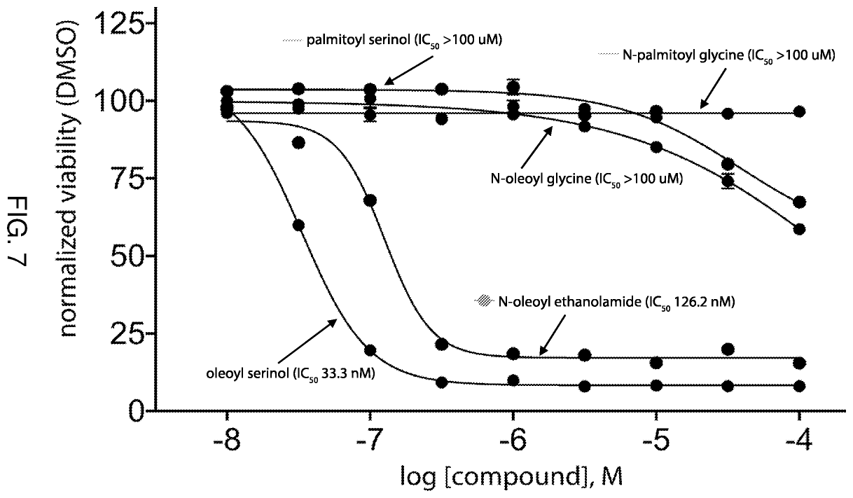

FIGS. 6B-6D are imaged liver sections stained with Sirius Red to identify collagen deposition, an indicator of liver fibrosis. Quantification of Sirius Red+ (collagen+) section area is shown in FIG. 6A. There was a statistically significant decrease in collagen deposition for N-acyl serinol treatment mice (Group I) as compared to control mice (Group III). There was no significant difference between point mutant mice (Group II) and control mice (Group III). Collagen quantification of Sirius Red stain was performed by morphometry with Bioquant™ software. All statistical analysis was carried out with Prism 9 software. Graph mean+/−s.e.m. *p<0.05. n=14 (Group I); n=9 (Group II); n=15 (Group III).

These data demonstrate mice receiving N-acyl serinol treatment exhibit less severe clinical symptoms of NAFLD, which suggests N-acyl serinol treatment may lessen the severity of and/or slow the progression of liver disease, and specifically, fibrosis and NASH (advanced liver damage and inflammation).

### Example 6: Generation of Tumor Organoids

Tumor organoids were prepared from the animal model described in Example 1 (i.e. tissue from control animals was isolated at 24 weeks and tumor organoids prepared therefrom). Tumor organoids were generated according to protocols known in the art (Broutier, L., Andersson-Rolf, A., Hindley, C J., Boj, S F., Clevers, H., et al., Culture and establishment of self-renewing human and mouse adult liver and pancreas 3D organoids and their genetic manipulation. Nat Protoc. 2016. 11(9): 1724-43 and Broutier, L. Mastrogiovanni, G., Verstegen, M M., Francies, H E., Gavarro, L M., et al., Human primary liver cancer-derived organoid cultures for disease modeling and drug screening. Nat Med. 2017 23(12): 1424-1435). Liver biopsies were digested in sterile PBS containing 0.125 mg/mL collagenase IV, 0.125 mg/mL dispase II, and 0.1 mg/mL DNaseI. Digestions were performed at 37° C. for at least 4 hours. Tissue dissociate was filtered through a 70 μm strainer and washed with basal media (Advanced DMEM/F12, 1% L-glutamine, 1% penstrep, 1 mM HEPES). Cells were counted, washed, and resuspended at 50,000 cells/50 μL matrigel. 50 μL matrigel droplets were plated in 24-well plates and allowed to polymerize for 15 minutes at 37° C. Tumor dissociate was cultured in tumor media (basal media, 1:50 B27, 1 mM N-acetylcysteine, 10 mM nicotinamide, 10 nM recombinant human [Leu¹⁵]-gastrin I, 50 ng/mL recombinant murine EGF, 100 ng/mL recombinant human FGF10, and 50 ng/mL recombinant human HGF) until organoids formed. To passage, organoids were removed from matrigel in basal media, spun down at 300 g for 5 minutes, mechanically broken via passage through a 21 g needle, washed in basal media, and re-plated in matrigel.

### Example 7: Treatment of Tumor Organoids with N-Acyl Amides

96-wells plates were coated with a 50:50 solution of matrigel:basal media, which was allowed to polymerize for 15 minutes at 37° C. Tumor organoids were mechanically broken as described above, counted, and seeded at 1,000 cells/well in tumor media. Following overnight incubation, serial drug dilutions were added to the tumor organoids. Drug treatment included palmitoyl serinol (IC₅₀>100 μM), N-oleoyl ethanolamide (IC₅₀=126.2 nM), N-oleoyl serinol (IC₅₀=33.3 nM), N-oleoyl glycine (IC₅₀>100 μM), and N-palmitoyl glycine (IC₅₀>100 PM). End-point viability was analyzed 3-days post-drug addition using CellTiter-Glo. Each treatment was performed in triplicate.

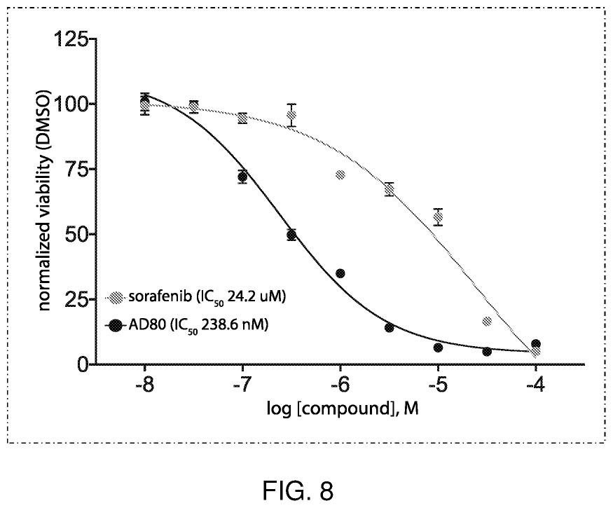

FIG. 7 illustrates dose-response curves for each drug treatment. FIG. 8 illustrates dose-response curves for Sorafenib (an FDA-approved small molecule drug for the treatment of hepatocellular carcinoma) and AD80 (a small molecule identified in a library screen for liver cancer inhibitors). These data demonstrate N-oleoyl serinol and its structurally related compound oleoylethanolamide inhibit or reduce liver cancer growth and/or survival, suggesting N-oleoyl serinol or N-oleoyl serinol and oleoylethanolamide can function as anti-tumor compounds for treatment of hepatocellular carcinoma. Graphs mean+/−s.e.m.

### Example 8: Generation and Treatment of Human Hepatocellular Cancer (HCC) Organoids

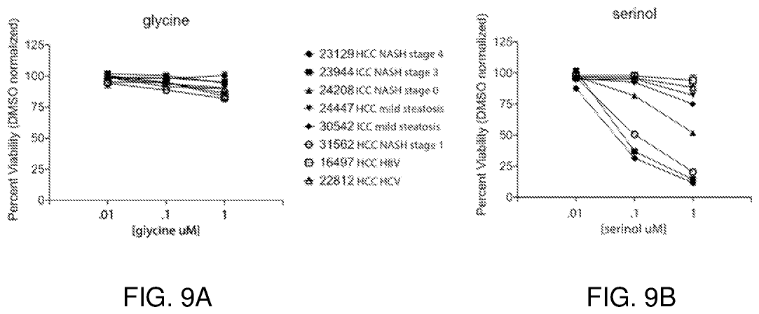

Human hepatocellular cancer (HCC) organoids were prepared from patients with non alcoholic steatohpepatitis (NASH), hepatitis B virus (HBV) and hepatitis C virus (HCV). The HCC organoids were generated according to protocols known in the art (Broutier, L., Andersson-Rolf, A., Hindley, C J., Boj, S F., Clevers, H., et al., Culture and establishment of self-renewing human and mouse adult liver and pancreas 3D organoids and their genetic manipulation. Nat Protoc. 2016. 11(9): 1724-43 and Broutier, L. Mastrogiovanni, G., Verstegen, M M., Francies, H E., Gavarro, L M., et al., Human primary liver cancer-derived organoid cultures for disease modeling and drug screening. Nat Med. 2017 23(12): 1424-1435). Organoids were also prepared from a single patient with cholangiocarcinoma (ICC). N-oleoyl serinol and N-oleoyl glycine were assayed for anti-tumor effects. Each treatment was performed in triplicate. As shown in FIGS. 9A and 9B, N-oleoyl glycine was inactive against all organoid cell lines. N-oleoyl serinol was active specifically against HCCC organoids from patients with NASH.

It will be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating adenocarcinoma in a subject, the method comprising administering to the subject in need thereof an N-acyl amide having Formula (1): 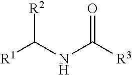
wherein R¹ is selected from the group consisting of carboxylate and CH₂OH;
R² is selected from the group consisting of H, (C₃-C₄) alkyl-NH₃⁺, (C₃-C₄)alkyl-NH₂, C₂ alkyl-C(═O)NH₂, CH₂OH, and methyl; and
R³ is selected from the group consisting of (C₉-C₁₈)alkyl, (C₉-C₁₈)alkenyl, wherein the (C₉-C₁₈)alkyl and (C₉-C₁₈)alkenyl are optionally substituted.

2. The method of claim 1, wherein Formula (1) of the N-acyl amide is represented by one of Formulae (2)-(6): 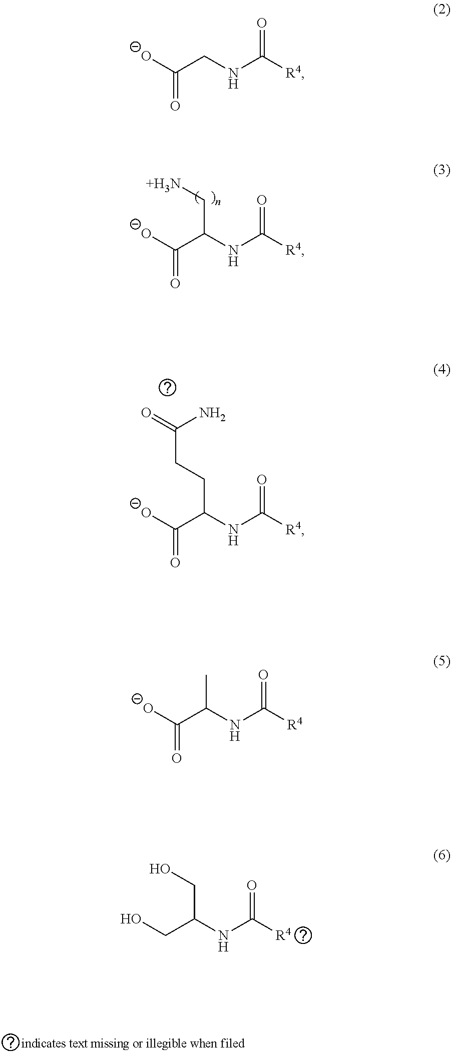
wherein R⁴ is selected from the group consisting of (C₉-C₁₈)alkyl, (C₉-C₁₈)alkenyl, wherein the (C₉-C₁₈)alkyl and (C₉-C₁₈)alkenyl are optionally substituted; and
n is 3 or 4.

3. The method of claim 2, wherein Formulae (2)-(6) are represented by Formulae (7)-(11): 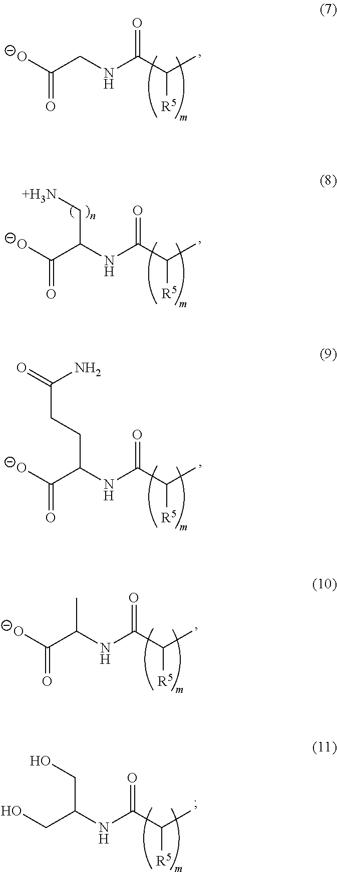
wherein R⁵ is independently selected from the group consisting of H and —OH; and
m is an integer from 8 to 17.

4. The method of claim 2, wherein Formulae (2)-(6) are represented by Formulae (12)-(16): 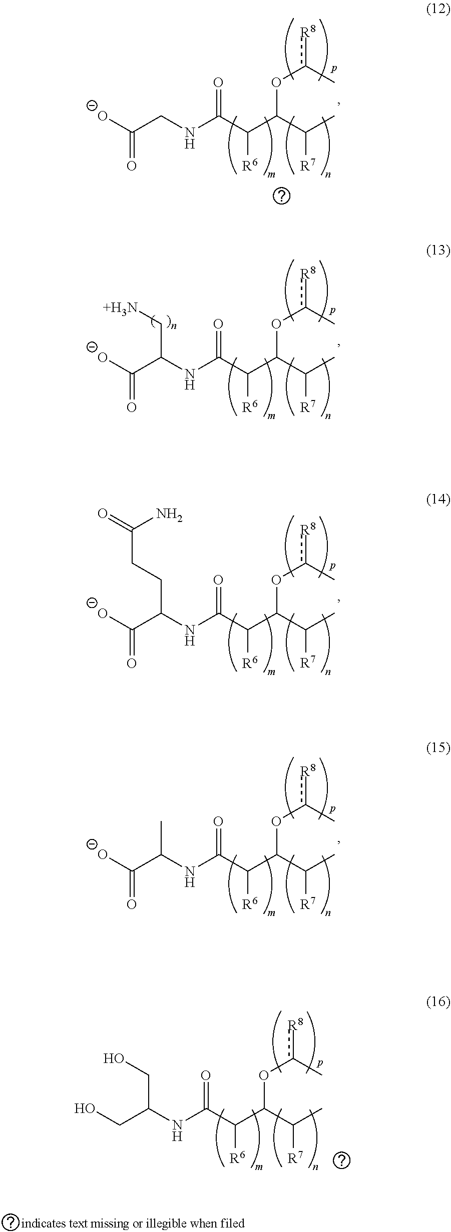
wherein R⁶, R⁷, and R⁸ are independently selected from the group consisting of H, —OH, and ═O;
m is an integer from 1 to 5;
n is an integer from 2 to 15;
p is an integer from 8 to 18; and
q is an integer from 3 to 4.

5. The method of claim 1, wherein the N-acyl amide is selected from the group consisting of:

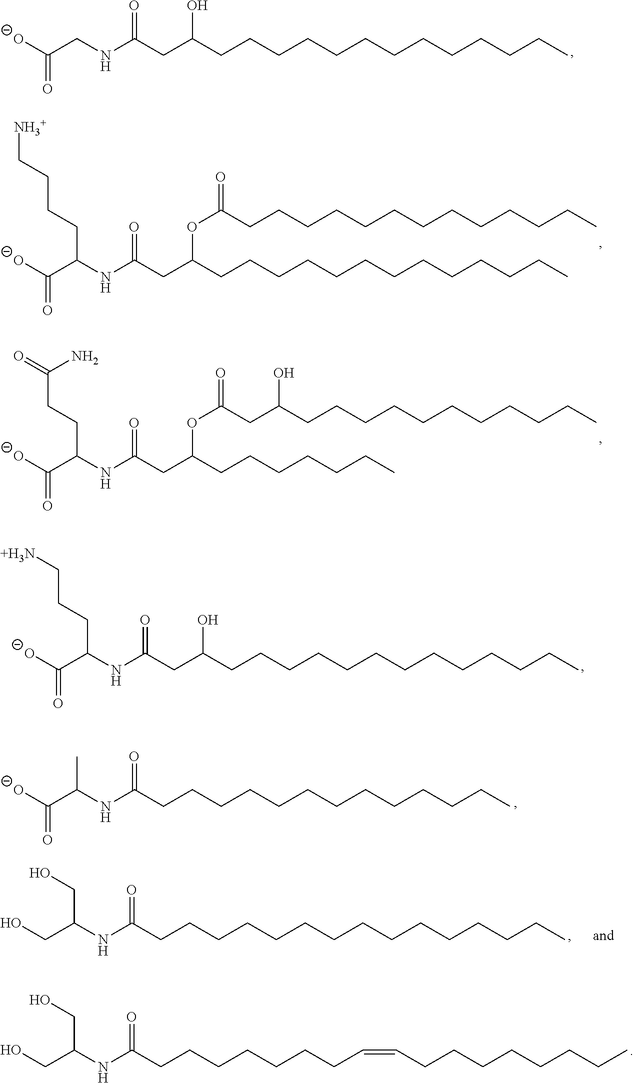

6. The method of claim 1, wherein the N-acyl amide is N-oleoyl serinol.

7. The method of claim 1, wherein the adenocarcinoma is found in the digestive system of the subject.

8. The method of claim 1, wherein the adenocarcinoma is found in the liver, pancreas, small intestine, large intestine, colon, or stomach.

9. The method of claim 1, wherein the adenocarcinoma is hepatocellular carcinoma.

10. A method of treating adenocarcinoma in a subject, the method comprising administering to the subject in need thereof a composition comprising at least one of a genetically engineered cell expressing a human microbial N-acyl synthase (hm-NAS) gene, a hm-NAS gene, or an N-acyl amide.

11. The method of claim 10, wherein the genetically engineered cell encodes an N-acyl synthase polypeptide that catalyzes synthesis of an N-acyl amide.

12. The method of claim 10, wherein the genetically engineered cell is a non-pathogenic bacterial cell.

13. The method of claim 12, wherein the non-pathogenic bacterial cell is E. coli.

14. The method of claim 10, wherein the hm-NAS gene is selected from the group consisting of EFI7261; EHB91285; EEK17761; EEY82825; EHP49568; EHG23013; EFA42931; EFL47029; EH075052; ADK95845; EFV04460; EHH01788; EDY97076; CBW20928; EDS14876; EDO52243; CBK67812; AC109609; ABV66681; EHT12133; EFE54303; EFE94777; EER56350; EET45812; ACS62992; BAH33083; EFG73978; CAW29482; EFH13337; EGP09383; EEV22085; EEY94333; EFF83269; CAP01857; EGP10046; EFK33376; EEK14630; EFS97491; CBK85930; EHM48796; EEK89350; EHL05550; EFV76279; GL883582; R6A3N1_9BACT/51-156; R6EH40_9BACT/51-155; R7PBT6_9BACT/52-156; R7NN97 9BACE/51-155; AOAOC3RD59_9PORP/51-157; A6L081 BACV8/51-155; A6LEV2_PARD8/51-155; D41Mll 9BACT/57-158; D5EVS3_PRER2/52-157; D6D060 9BACE/51-155; E6SVIO_BACT6/51-155; CBK67812_CBK67812.l_Bacteroides_xylanisolvens_XB1A_hypothetical_protein; ENA_CBW20928_CBW20928.l_Bacteroides_fragilis_638R_putative_hemolysin_A; ENA_EDO52243 EDO52243.l_Bacteroides_uniformis_ATCC_8492_hemolysin; ENA_EDSI 4876_EDS 14876. l_Bacteroides_stercoris_ATCC_43183_hemolysin_; ENA_EDY97076_EDY97076.l_Bacteroides_plebeius_DSM_1 7135 hemolysin; ENA_EEY82825_EEY82825.l_Bacteroides_sp._2_1_33B_hemolysin_; ENA_EFV04460_EFV04460.l_Prevotella_salivae_DSM_15606_hemolysin; ENA_EHB91285_EHB91285.l Alistipes_indistinctus_YIT_12060_hypothetical_protein_; ENA_EHH01788_EHHIO1 788.l_Paraprevotella_clara_YIT 11 840_hemolysin; ENA_EHP49568_EHP49568.l_Odoribacter_laneus_YIT_12061_hypothetical_protein; l3YLB0_ALIFI/56-157; Q5LII1_BACFN/51-155; Q8A247 BACTN/51-155; R5C642 9BACE/51-155; R5FQF1_9BACT/53-157; R51942_9PORP/51-156; R5JGR8 9BACE/51-155; R5KD71 9BACT/52-157; R5MMX8 9BACE/51-155; R5NZI1 9BACT/51-155; R5UEV5 9BACE/51-155; R5UP15_9PORP/51-157; R5VW07_9BACE/51-155; R6B4U0_9BACT/52-156; R6BXV9 9BACT/52-157; R6DH15 9BACE/51-155; R6FKP1 9BACE/51-155; R6FUQ8_9BACT/52-158; R6KTM3 9BACE/51-155; R6LNJ9_9BACE/51-154; R6MX16 9BACE/51-155; R6QE29_9BACT/52-157; R6S950_9BACE/51-155; R6SC61_9BACE/51-155; R6VUA1_9BACT/56-157; R6XGV7 9BACT/52-157; R6YIB5_9BACE/51-155; R7DDR3 9PORP/51-155; R7EIP8_9BACE/51-155; R7F021_9BACT/51-157; R7HSG0_9BACT/37-143; R7IYP9_9BACT/59-165; R7JHM4_9BACT/51-152; E6K481 9BACT/52-156; ENA_ADK95 845 ADK95845.l_Prevotella_melaninogenica_ATCC_25 845 hemolysin; ENA_EFil 7261_EF!l 7261.l_Bacteroidetes_oral_taxon_274_str._F0058_hemolysin; ENA_EHG23013_EHG23013.l_Alloprevotella_rava_F0323_hypothetical_protein; ENA_EHO7 5052_EH075052.l_Prevotella_micans_F0438_hypothetical_protein; F2KX19_PREDF/64-168; F903S1_PREDD/52-156 1; 11 YUM9 PREl7/53-157; Q7MTR9_PORGV53-158; R5CSR0_9BACT/52-157; R5GFN8_9BACT/51-155; R5Q4D6_9BACT/52-157; R6W2Q2_9BACT/52-156; R7CYB8 9BACE/51-155; W0EP20 9PORP/51-155; C7M608_CAPOD/352-453; ENA_EEK14630_EEK14630.l_Capnocytophaga_gingivalis_ATCC_33624_Acyltransferase_; ENA_EFS97491_EFS97491.l_Capnocytophaga_ochracea_F0287_Acyltransferase; F9YU78 CAPCC/351-452; H1Z9S5 MYROD/346-447 ENA_EFA4293 l_EFA4293 l.l_Prevotella_bergensis_DSM_1 7361 hemolysin; A0A095ZG93 9BACT/52-156; E7RNE3 9BACT/52-156; ENA_EEKl 7761 EEK1 7761.l_Porphyromonas_uenonis_60-3_hemolysin_; ENA EFIA7029 EFL47029.1 Prevotella_disiens_FB035-09AN_hemolysin_; F4KL89_PORAD/55-160; 14Z8L9 9BACT/52-156; R6CE12 9BACE/51-155; R6XAK6_9BACT/52-156 ENA EHL05550 EHL05550.l_Desulfitobacterium_hafniense_DP7 aminotransferase class_V; ENA_EFV76279 EFV76279.l_Bacillus_sp._2_A_57 CT2_serinepyruvate_arninotransferase; A6T596_KLEP7/322-423; D8MWX6_ERWBE/367-468; ENA_EFE94777 EFE94 777.l_Serratia_odorifera_DSM_45 82_Acyltransferase; Q6CZN2_PECAS/322-423; A0A0B5CH45_NEIEG/32-132; E5UJR0_NEIMU/32-132; ENA_EET 45 812_EET 45 812.l_Neisseria_sicca_ATCC_29256_hypothetical_protein; ENA_ACI09609_ACI09609.l_Klebsiella_pneumoniae_342_conserved_hypothetical_protein; A4W746 ENT38/322-423; ENA_CBK85930_CBK85930.l_Enterobacter_cloacae_subsp._cloacae_NCTC_9394_Putative_h emolysin_; ENA_EFE54303_EFE54303.l_Providencia_rettgeri_DSM_ll3l_Acyltransferase; ENA_EHM48796_EHM48796. l_Yokenella_regensburgei_ATCC_43003_Acyltransferase; F9ZAJ4_ODOSD/341-443; G9Z3T1 9ENTR/322-423; R5UYM1_9PORP/338-439; ENA_ACS62992_ACS62992.l_Ralstonia_pickettii_12D_conserved_hypothetical_protein_ENA_CAW29482_CAW29482.l_Pseudomonas_aeruginosa_LESB58_putative_hemolysin; A0A089UDH2_9ENTR/323-424; E6WAC8_PANSA/322-423; ENA_EHT12133_EH T12133.l_Raoultella_omithinolytica_10-5246_hypothetical_protein; G7LV45_9EN TR/322-423; ENA_EER56350_EER56350.l_N eisseria_flavescens_SK1 1 4_hypothetical_protein_; AOA077KL19 9FLAO/353-454; A7MLT3_CROS8/322-423; ENA_EFK33376_EF K33376.1 Chryseobacterium gleum_ATCC_35910_Acyltransferase; and ENA_CAP01 857 CAP01857.2_Acinetobacter_baumannii_SDF_conserved_hypothetical_protein_.

15. The method of claim 14, wherein the hm-NAS gene is N-acyl serinol synthase.

16. The method of claim 10, wherein the N-acyl amide has Formula (1): 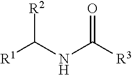
wherein R¹ is selected from the group consisting of carboxylate and CH₂OH;
R² is selected from the group consisting of H, (C₃-C₄) alkyl-NH₃⁺, (C₃-C₄)alkyl-NH₂, C₂ alkyl-C(═O)NH₂, CH₂OH, and methyl; and
R³ is selected from the group consisting of (C₉-C_{1g})alkyl, (C₉-C₁₈)alkenyl, wherein the (C₉-C₁₈)alkyl and (C₉-C₁₈)alkenyl are optionally substituted.

17. The method of claim 16, wherein Formula (1) of the N-acyl amide is represented by one of Formulae (2)-(6): 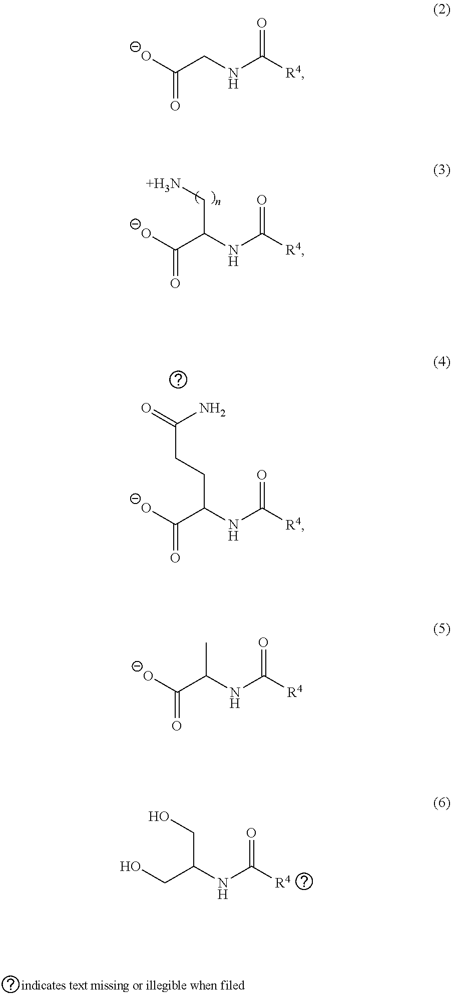
wherein R⁴ is selected from the group consisting of (C₉-C₁₈)alkyl, (C₉-C₁₈)alkenyl, wherein the (C₉-C₁₈)alkyl and (C₉-C₁₈)alkenyl are optionally substituted; and
n is 3 or 4.

18. The method of claim 17, wherein Formulae (2)-(6) are represented by Formulae (7)-(11): 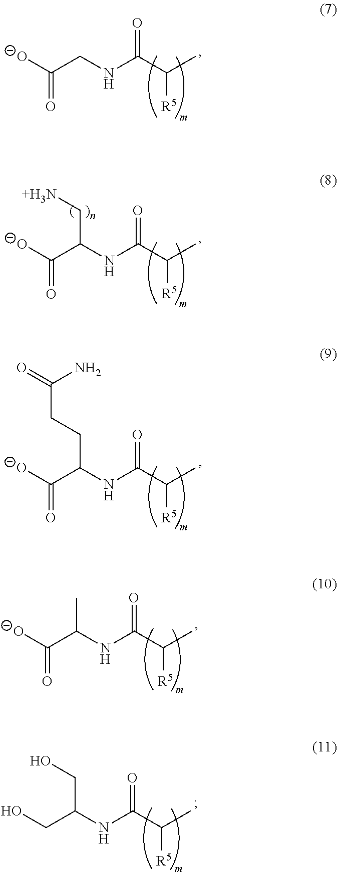
wherein R⁵ is independently selected from the group consisting of H and —OH; and
m is an integer from 8 to 17.

19. The method of claim 17, wherein Formulae (2)-(6) are represented by Formulae (12)-(16): 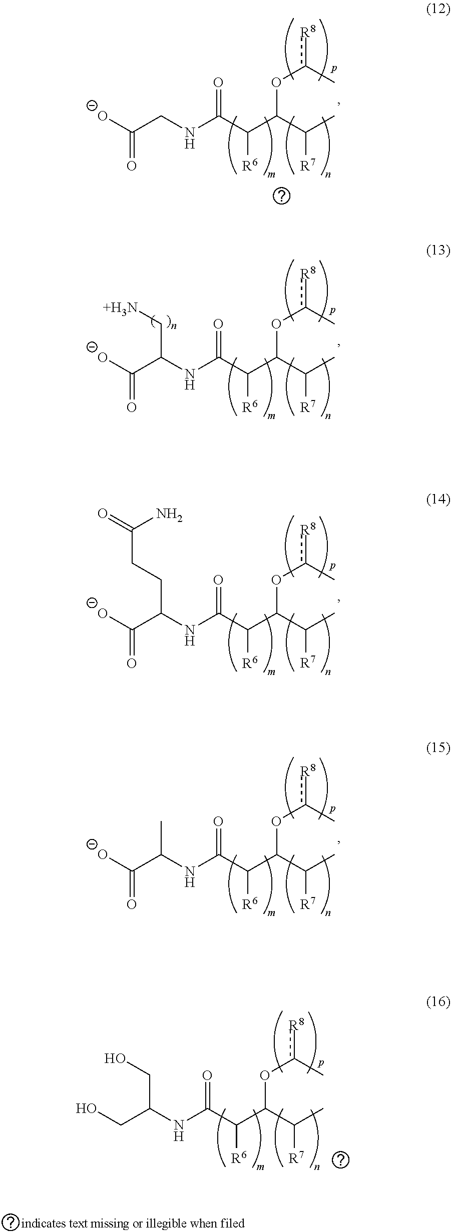
wherein R⁶, R⁷, and R⁸ are independently selected from the group consisting of H, —OH, and ═O;
m is an integer from 1 to 5;
n is an integer from 2 to 15;
p is an integer from 8 to 18; and
q is an integer from 3 to 4.

20. The method of claim 16, wherein the N-acyl amide is selected from the group consisting of.

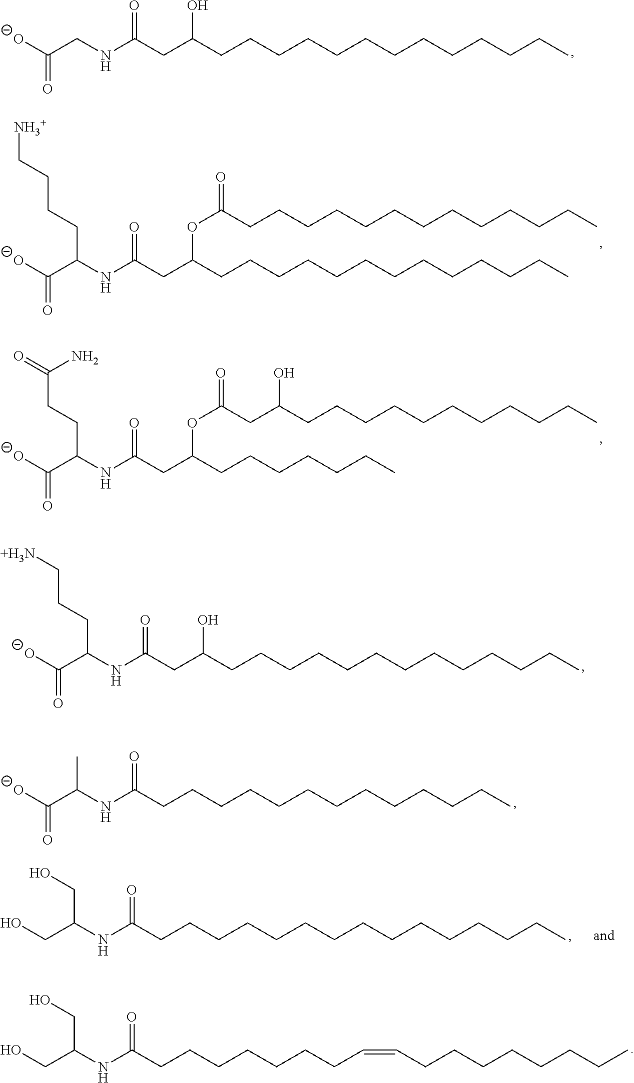

21. The method of claim 16, wherein the N-acyl amide is wherein the N-acyl amide is N-oleoyl serinol.

22. The method of claim 10, wherein the composition is administered in a therapeutically effective amount.

23. The method of claim 10, wherein the composition further comprises a pharmaceutically acceptable carrier, diluent, buffer, or excipient.

24. The method of claim 10, wherein the adenocarcinoma is found in the digestive system of the subject.

25. The method of claim 10, wherein the adenocarcinoma is found in the liver, pancreas, small intestine, large intestine, colon, or stomach.

26. The method of claim 10, wherein the adenocarcinoma is hepatocellular carcinoma.

27. A method of treating liver cancer in a subject, the method comprising administering to the subject in need thereof a composition comprising at least one of a genetically engineered cell expressing a human microbial N-acyl synthase (hm-NAS) gene, a hm-NAS gene, or an N-acyl amide.

28. The method of claim 27, wherein the genetically engineered cell encodes an N-acyl synthase polypeptide that catalyzes synthesis of an N-acyl amide.

29. The method of claim 27, wherein the genetically engineered cell is a non-pathogenic bacterial cell.

30. The method of claim 29, wherein the non-pathogenic bacterial cell is E. coli.

31. The method of claim 27, wherein the hm-NAS gene is selected from the group consisting of EFI7261; EHB91285; EEK17761; EEY82825; EHP49568; EHG23013; EFA42931; EFL47029; EH075052; ADK95845; EFV04460; EHH01788; EDY97076; CBW20928; EDS14876; EDO52243; CBK67812; AC109609; ABV66681; EHT12133; EFE54303; EFE94777; EER56350; EET45812; ACS62992; BAH33083; EFG73978; CAW29482; EFH13337; EGP09383; EEV22085; EEY94333; EFF83269; CAP01857; EGP10046; EFK33376; EEK14630; EFS97491; CBK85930; EHM48796; EEK89350; EHL05550; EFV76279; GL883582; R6A3N1_9BACT/51-156; R6EH40_9BACT/51-155; R7PBT6_9BACT/52-156; R7NN97 9BACE/51-155; AOAOC3RD59_9PORP/51-157; A6L081 BACV8/51-155; A6LEV2_PARD8/51-155; D41Mll 9BACT/57-158; D5EVS3_PRER2/52-157; D6D060 9BACE/51-155; E6SVIO_BACT6/51-155; CBK67812_CBK67812.l_Bacteroides_xylanisolvens_XB1A_hypothetical_protein; ENA_CBW20928_CBW20928.l_Bacteroides_fragilis_638R_putative_hemolysin_A; ENA_ED052243 ED052243.l_Bacteroides_uniformis_ATCC_8492_hemolysin; ENA_EDS1 4876_EDS 14876.l_Bacteroides_stercoris_ATCC_43183_hemolysin; ENA_EDY97076_EDY97076.l_Bacteroides_plebeius_DSM_1 7135 hemolysin; ENA_EEY82825_EEY82825.l_Bacteroides_sp._2_1_33B_hemolysin_; ENA_EFV04460_EFV04460.l_Prevotella_salivae_DSM_15606_hemolysin; ENA_EHB91285_EHB91285.1 Alistipes_indistinctus_YIT_12060_hypothetical_protein_; ENA_EHH01788_EHHIO1 788.l_Paraprevotella_clara_YIT 11 840_hemolysin; ENA_EHP49568_EHP49568.l_Odoribacter_laneus_YIT_12061_hypothetical_protein; 13YLB0_ALIFI/56-157; Q5LII1_BACFN/51-155; Q8A247 BACTN/51-155; R5C642 9BACE/51-155; R5FQF1_9BACT/53-157; R51942_9PORP/51-156; R5JGR8 9BACE/51-155; R5KD71 9BACT/52-157; R5MMX8 9BACE/51-155; R5NZI1 9BACT/51-155; R5UEV5 9BACE/51-155; R5UP15_9PORP/51-157; R5VW07_9BACE/51-155; R6B4U0_9BACT/52-156; R6BXV9 9BACT/52-157; R6DH15 9BACE/51-155; R6FKP1 9BACE/51-155; R6FUQ8_9BACT/52-158; R6KTM3 9BACE/51-155; R6LNJ9_9BACE/51-154; R6MX16 9BACE/51-155; R6QE29_9BACT/52-157; R6S950_9BACE/51-155; R6SC61_9BACE/51-155; R6VUA1_9BACT/56-157; R6XGV7 9BACT/52-157; R6YIB5_9BACE/51-155; R7DDR3 9PORP/51-155; R7EIP8_9BACE/51-155; R7F021_9BACT/51-157; R7HSG0_9BACT/37-143; R7IYP9_9BACT/59-165; R7JHM4_9BACT/51-152; E6K481 9BACT/52-156; ENA_ADK95 845 ADK95845.l_Prevotella_melaninogenica_ATCC_25 845 hemolysin; ENA_EFil 7261_EF!l 7261.l_Bacteroidetes_oral_taxon_274_str._F0058_hemolysin; ENA_EHG23013_EHG23013. l_Alloprevotella_rava_F0323 hypothetical protein; ENA_EHO7 5052_EHO75052.l_Prevotella_micans_F0438_hypothetical_protein; F2KX19_PREDF/64-168; F903S1_PREDD/52-156 1; 11 YUM9 PREl7/53-157; Q7MTR9_PORGV53-158; R5CSR0_9BACT/52-157; R5GFN8_9BACT/51-155; R5Q4D6_9BACT/52-157; R6W2Q2_9BACT/52-156; R7CYB8 9BACE/51-155; W0EP20 9PORP/51-155; C7M608_CAPOD/352-453; ENA_EEK14630_EEK14630.l_Capnocytophaga_gingivalis_ATCC_33624_Acyltransferase_; ENA_EFS97491_EFS97491.l_Capnocytophaga_ochracea_F0287_Acyltransferase; F9YU78_CAPCC/351-452; H1Z9S5 MYROD/346-447 ENA_EFA42931_EFA4293 l.l_Prevotella_bergensis_DSM_1 7361 hemolysin; A0A095ZG93 9BACT/52-156; E7RNE3 9BACT/52-156; ENA_EEKl 7761 EEK1 7761.l_Porphyromonas_uenonis_60-3_hemolysin_; ENA EFIA7029 EFL47029.1 Prevotella_disiens_FB035-09AN_hemolysin_; F4KL89_PORAD/55-160; 14Z8L9 9BACT/52-156; R6CE12 9BACE/51-155; R6XAK6_9BACT/52-156 ENA EHL05550 EHL05550.l_Desulfitobacterium_hafniense_DP7 aminotransferase class_V; ENA_EFV76279 EFV76279.l_Bacillus_sp._2_A_57 CT2_serinepyruvate_arninotransferase; A6T596_KLEP7/322-423; D8MWX6_ERWBE/367-468; ENA_EFE94777 EFE94 777.l_Serratia_odorifera_DSM_45 82_Acyltransferase; Q6CZN2_PECAS/322-423; AOAOB5CH45_NEIEG/32-132; E5UJR0_NEIMU/32-132; ENA_EET 45 812_EET 45 812.l_Neisseria_sicca_ATCC_29256_hypothetical_protein; ENA_ACI09609_ACI09609.l_Klebsiella_pneumoniae_342_conserved_hypothetical_protein; A4W746 ENT38/322-423; ENA_CBK85930_CBK85930.l_Enterobacter_cloacae_subsp._cloacae_NCTC_9394_Putative_h emolysin_; ENA_EFE54303_EFE54303.l_Providencia_rettgeri_DSM_ll3l_Acyltransferase; ENA_EHM48796_EHM48796. l_Yokenella_regensburgei_ATCC_43003_Acyltransferase; F9ZAJ4 ODOSD/341-443; G9Z3Tl 9ENTR/322-423; R5UYM1_9PORP/338-439; ENA_ACS62992_ACS62992.l_Ralstonia_pickettii_12D_conserved_hypothetical_protein_ENA_CAW29482_CAW29482.l_Pseudomonas_aeruginosa_LESB58_putative_hemolysin; AOA089UDH2_9ENTR/323-424; E6WAC8_PANSA/322-423; ENA_EHT12133_EH T12133.l_Raoultella_omithinolytica_10-5246_hypothetical_protein; G7LV45_9EN TR/322-423; ENA_EER56350_EER56350.l_N eisseria_flavescens_SK11 4_hypothetical_protein_; AOA077KL19 9FLAO/353-454; A7MLT3_CROS8/322-423; ENA_EFK33376_EF K33376.l_Chryseobacterium_gleum_ATCC_35910_Acyltransferase_; and ENA_CAP01 857 CAP01857.2_Acinetobacter_baumannii_SDF_conserved_hypothetical_protein_.

32. The method of claim 31, wherein the hm-NAS gene is N-acyl serinol synthase.

33. The method of claim 27, wherein the N-acyl amide has Formula (1): 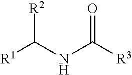
wherein R¹ is selected from the group consisting of carboxylate and CH₂OH;
R² is selected from the group consisting of H, (C₃-C₄) alkyl-NH₃⁺, (C₃-C₄)alkyl-NH₂, C₂ alkyl-C(═O)NH₂, CH₂OH, and methyl; and
R³ is selected from the group consisting of (C₉-C₁₈)alkyl, (C₉-C₁₈)alkenyl, wherein the (C₉-C₁₈)alkyl and (C₉-C₁₈)alkenyl are optionally substituted.

34. The method of claim 33, wherein Formula (1) of the N-acyl amide is represented by one of Formulae (2)-(6): 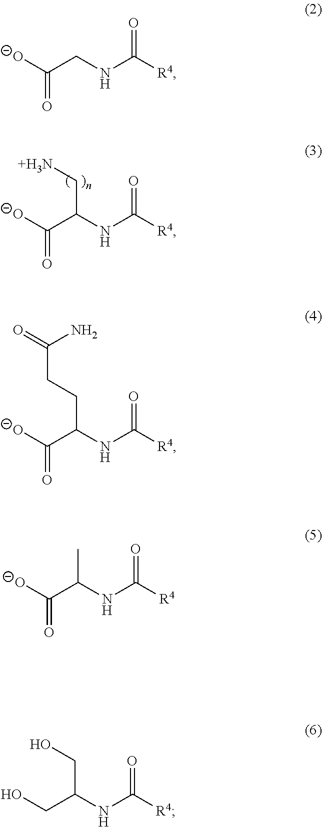
wherein R⁴ is selected from the group consisting of (C₉-C₁₈)alkyl, (C₉-C₁₈)alkenyl, wherein the (C₉-C₁₈)alkyl and (C₉-C₁₈)alkenyl are optionally substituted; and
n is 3 or 4.

35. The method of claim 34, wherein Formulae (2)-(6) are represented by Formulae (7)-(11): 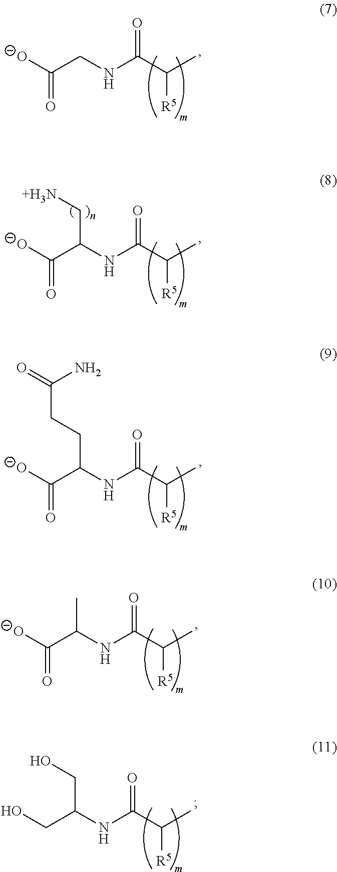
wherein R⁵ is independently selected from the group consisting of H and —OH; and
m is an integer from 8 to 17.

36. The method of claim 34, wherein Formulae (2)-(6) are represented by Formulae (12)-(16): 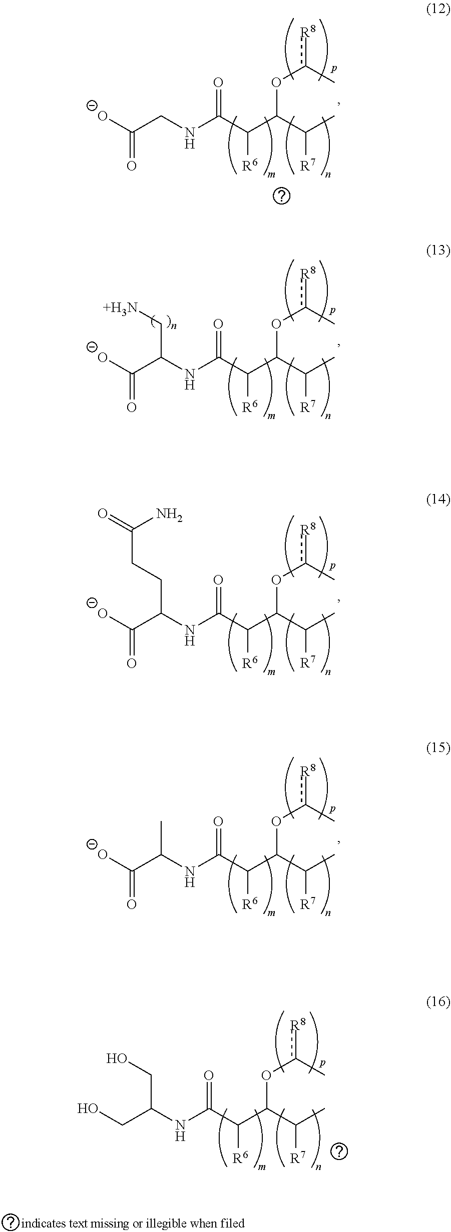
wherein R⁶, R⁷, and R⁸ are independently selected from the group consisting of H, —OH, and ═O;
m is an integer from 1 to 5;
n is an integer from 2 to 15;
p is an integer from 8 to 18; and
q is an integer from 3 to 4.

37. The method of claim 33, wherein the N-acyl amide is selected from the group consisting of:

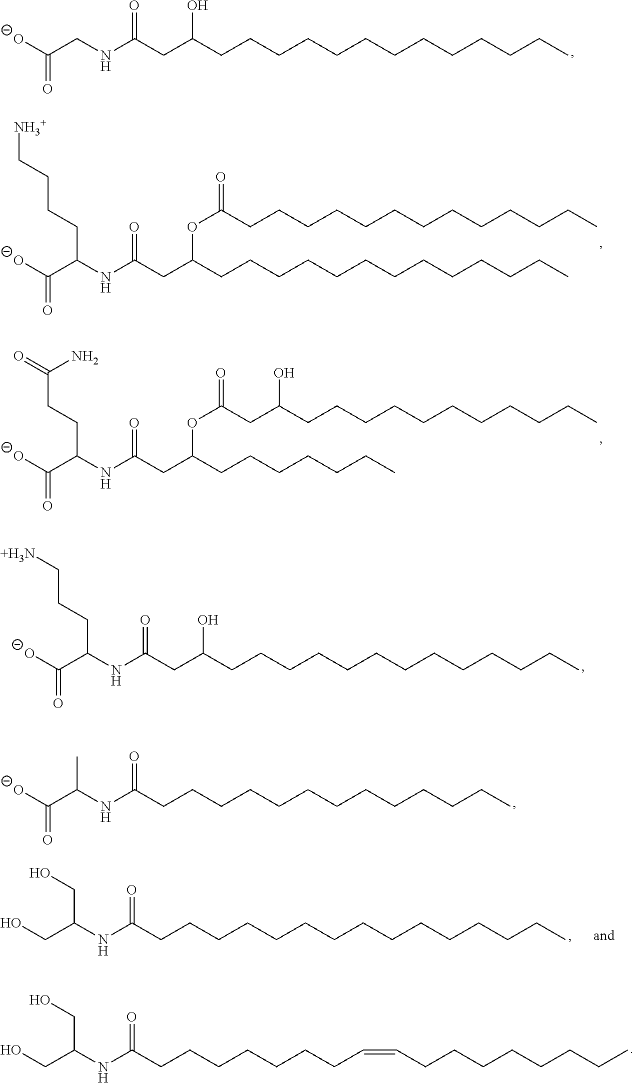

38. The method of claim 33, wherein the N-acyl amide is wherein the N-acyl amide is N-oleoyl serinol.

39. The method of claim 27, wherein the composition is administered in a therapeutically effective amount.

40. The method of claim 27, wherein the composition further comprises a pharmaceutically acceptable carrier, diluent, buffer, or excipient.

41. The method of claim 27, wherein the liver cancer is hepatocellular carcinoma.

42. A method of treating adenocarcinoma in a subject using a live biotherapeutic, the method comprising administering to the subject in need thereof a composition comprising a genetically engineered cell expressing a human microbial N-acyl synthase (hm-NAS) gene, wherein the hm-NAS gene encodes an N-acyl synthase polypeptide.

43. The method of claim 42, wherein the N-acyl synthase polypeptide catalyzes synthesis of an N-acyl amide.

44. The method of claim 42, wherein the genetically engineered cell is a non-pathogenic bacterial cell.

45. The method of claim 44, wherein the non-pathogenic bacterial cell is E. coli.

46. The method of claim 42, wherein the hm-NAS gene is selected from the group consisting of EFI7261; EHB91285; EEK17761; EEY82825; EHP49568; EHG23013; EFA42931; EFL47029; EH075052; ADK95845; EFV04460; EHH01788; EDY97076; CBW20928; EDS14876; EDO52243; CBK67812; AC109609; ABV66681; EHT12133; EFE54303; EFE94777; EER56350; EET45812; ACS62992; BAH33083; EFG73978; CAW29482; EFH13337; EGP09383; EEV22085; EEY94333; EFF83269; CAP01857; EGP10046; EFK33376; EEK14630; EFS97491; CBK85930; EHM48796; EEK89350; EHL05550; EFV76279; GL883582; R6A3N1_9BACT/51-156; R6EH40_9BACT/51-155; R7PBT6_9BACT/52-156; R7NN97 9BACE/51-155; AOAOC3RD59_9PORP/51-157; A6L081 BACV8/51-155; A6LEV2_PARD8/51-155; D41Mll 9BACT/57-158; D5EVS3_PRER2/52-157; D6D060 9BACE/51-155; E6SVIO_BACT6/51-155; CBK67812_CBK67812.l_Bacteroides_xylanisolvens_XB1A_hypothetical_protein; ENA_CBW20928_CBW20928.l_Bacteroides_fragilis_638R_putative_hemolysin_A; ENA_EDO52243 EDO52243. l_Bacteroides_uniformis_ATCC_8492_hemolysin; ENA_EDSl 4876_EDS 14876. l_Bacteroides_stercoris_ATCC_43183_hemolysin; ENA_EDY97076_EDY97076. l_Bacteroides_plebeius_DSM_1 7135 hemolysin; ENA_EEY82825_EEY82825. l_Bacteroides_sp._2_1_33B_hemolysin_; ENA_EFV04460_EFV04460. l_Prevotella_salivae_DSM_15606_hemolysin; ENA_EHB91285_EHB91285.l_Alistipes_indistinctus_YIT_12060_hypothetical_protein_ENA_EHH01788_EHHO1 788. l_Paraprevotella_clara_YIT 11840_hemolysin; ENA_EHP49568_EHP49568. l_Odoribacter_laneus_YIT_12061_hypothetical_protein; 13YLB0_ALIFI/56-157; Q5LII1_BACFN/51-155; Q8A247 BACTN/51-155; R5C642 9BACE/51-155; R5FQF1_9BACT/53-157; R51942_9PORP/51-156; R5JGR8 9BACE/51-155; R5KD71 9BACT/52-157; R5MMX8 9BACE/51-155; R5NZI1 9BACT/51-155; R5UEV5 9BACE/51-155; R5UP15_9PORP/51-157; R5VW07_9BACE/51-155; R6B4U0_9BACT/52-156; R6BXV9 9BACT/52-157; R6DH15 9BACE/51-155; R6FKP1 9BACE/51-155; R6FUQ8_9BACT/52-158; R6KTM3 9BACE/51-155; R6LNJ9_9BACE/51-154; R6MX16 9BACE/51-155; R6QE29_9BACT/52-157; R6S950_9BACE/51-155; R6SC61_9BACE/51-155; R6VUA1_9BACT/56-157; R6XGV7 9BACT/52-157; R6YIB5_9BACE/51-155; R7DDR3 9PORP/51-155; R7EIP8_9BACE/51-155; R7F021_9BACT/51-157; R7HSG0_9BACT/37-143; R7IYP9_9BACT/59-165; R7JHM4_9BACT/51-152; E6K481 9BACT/52-156; ENA_ADK95 845 ADK95845.l_Prevotella_melaninogenica_ATCC_25 845 hemolysin; ENA_EFil 7261_EF!l 7261.l_Bacteroidetes_oral_taxon_274_str._F0058_hemolysin; ENA_EHG23013_EHG23013.l_Alloprevotella_rava_F0323_hypothetical_protein; ENA_EHO7 5052_EH075052.l_Prevotella_micans_F0438_hypothetical_protein; F2KX19_PREDF/64-168; F903S1_PREDD/52-156 1; 11 YUM9 PREl7/53-157; Q7MTR9_PORGV53-158; R5CSR0_9BACT/52-157; R5GFN8_9BACT/51-155; R5Q4D6_9BACT/52-157; R6W2Q2_9BACT/52-156; R7CYB8 9BACE/51-155; W0EP20 9PORP/51-155; C7M608_CAPOD/352-453; ENA_EEK14630_EEK14630.1 Capnocytophaga gingivalis_ATCC_33624 Acyltransferase_: ENA_EFS97491_EFS97491.l_Capnocytophaga_ochracea_F0287_Acyltransferase; F9YU78_CAPCC/351-452; H1Z9S5 MYROD/346-447 ENA_EFA4293 l_EFA4293 l.l_Prevotella_bergensis_DSM_1 7361 hemolysin; A0A095ZG93 9BACT/52-156; E7RNE3 9BACT/52-156; ENA_EEKl 7761_EEK1 7761.l_Porphyromonas_uenonis_60-3_hemolysin_; ENA EFIA7029 EFL47029.1 Prevotella_disiens_FB035-09AN_hemolysin_; F4KL89_PORAD/55-160; 14Z8L9 9BACT/52-156; R6CE12 9BACE/51-155; R6XAK6_9BACT/52-156 ENA EHL05550 EHL05550.l_Desulfitobacterium_hafniense_DP7 aminotransferase class_V; ENA_EFV76279 EFV76279.l_Bacillus_sp._2_A_57 CT2_serinepyruvate_arninotransferase; A6T596_KLEP7/322-423; D8MWX6_ERWBE/367-468; ENA_EFE94777 EFE94 777.l_Serratia_odorifera_DSM_45 82_Acyltransferase; Q6CZN2_PECAS/322-423; A0A0B5CH45_NEIEG/32-132; E5UJR0 NEIMU/32-132; ENA_EET 45 812 EET 45 812. l_Neisseria_sicca_ATCC_29256_hypothetical_protein; ENA_ACI09609_ACI09609.l_Klebsiella_pneumoniae_342_conserved_hypothetical_protein; A4W746 ENT38/322-423; ENA_CBK85930_CBK85930.l_Enterobacter_cloacae_subsp._cloacae_NCTC_9394_Putative_h emolysin_; ENA_EFE54303_EFE54303.l_Providencia_rettgeri_DSM_ll3l_Acyltransferase; ENA_EHM48796_EHM48796. l_Yokenella_regensburgei_ATCC_43003_Acyltransferase; F9ZAJ4_ODOSD/341-443; G9Z3T1 9ENTR/322-423; R5UYM1_9PORP/338-439; ENA_ACS62992_ACS62992.l_Ralstonia_pickettii_12D_conserved_hypothetical_protein_ENA_CAW29482_CAW29482.l_Pseudomonas_aeruginosa_LESB58_putative_hemolysin; A0A089UDH2_9ENTR/323-424; E6WAC8_PANSA/322-423; ENA_EHT12133_EH T12133.1 Raoultella_omithinolytica_10-5246 hypothetical_protein; G7LV45_9EN TR/322-423; ENA_EER56350_EER56350. l_N eisseria_flavescens_SK1 1 4_hypothetical_protein_; AOA077KL19 9FLAO/353-454; A7MLT3_CROS8/322-423; ENA_EFK33376_EF K33376.l_Chryseobacterium_gleum_ATCC_35910_Acyltransferase_; and ENA_CAPO1 857 CAP01857.2_Acinetobacter_baumannii_SDF conserved_hypothetical_protein_.

47. The method of claim 46, wherein the hm-NAS gene is N-acyl serinol synthase.

48. The method of claim 43, wherein the N-acyl amide has Formula (1): 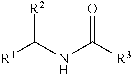
wherein R¹ is selected from the group consisting of carboxylate and CH₂OH;
R² is selected from the group consisting of H, (C₃-C₄) alkyl-NH₃⁺, (C₃-C₄)alkyl-NH₂, C₂ alkyl-C(═O)NH₂, CH₂OH, and methyl; and
R³ is selected from the group consisting of (C₉-C₁₈)alkyl, (C₉-C₁₈)alkenyl, wherein the (C₉-C₁₈)alkyl and (C₉-C₁₈)alkenyl are optionally substituted.

49. The method of claim 48, wherein Formula (1) of the N-acyl amide is represented by one of Formulae (2)-(6): 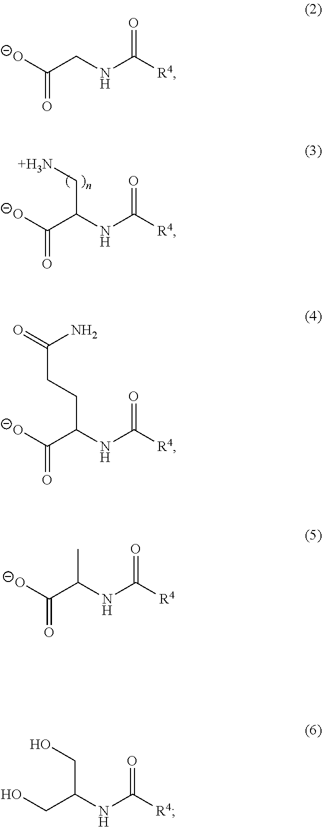
wherein R⁴ is selected from the group consisting of (C₉-C₁₈)alkyl, (C₉-C₁₈)alkenyl, wherein the (C₉-C₁₈)alkyl and (C₉-C₁₈)alkenyl are optionally substituted; and
n is 3 or 4.

50. The method of claim 49, wherein Formulae (2)-(6) are represented by Formulae (7)-(11): 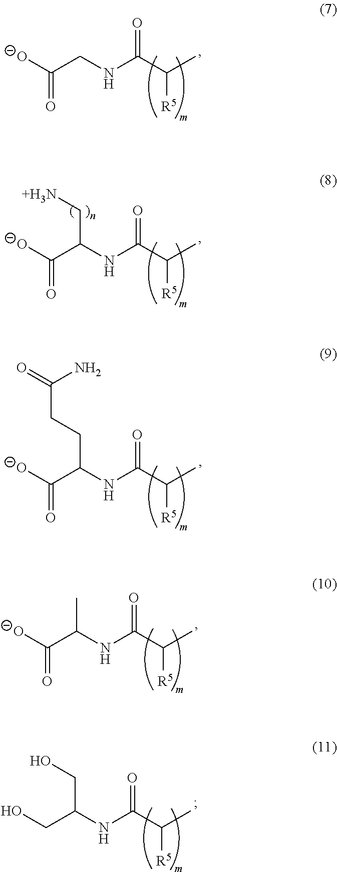
wherein R⁵ is independently selected from the group consisting of H and —OH; and
m is an integer from 8 to 17.

51. The method of claim 49, wherein Formulae (2)-(6) are represented by 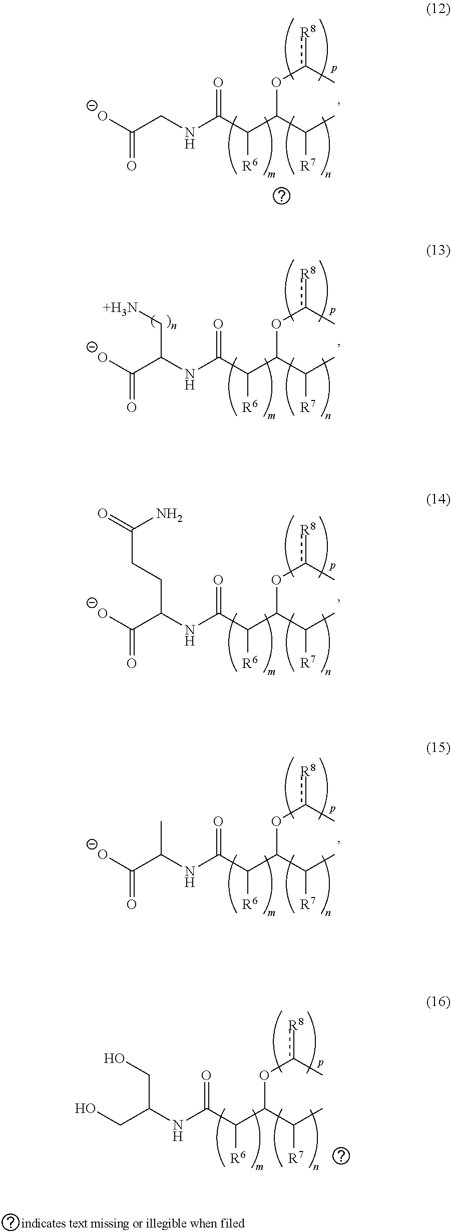
wherein R⁶, R⁷, and R⁸ are independently selected from the group consisting of H, —OH, and ═O;
m is an integer from 1 to 5;
n is an integer from 2 to 15;
p is an integer from 8 to 18; and
q is an integer from 3 to 4.

52. The method of claim 48, wherein the N-acyl amide is selected from the group consisting of:

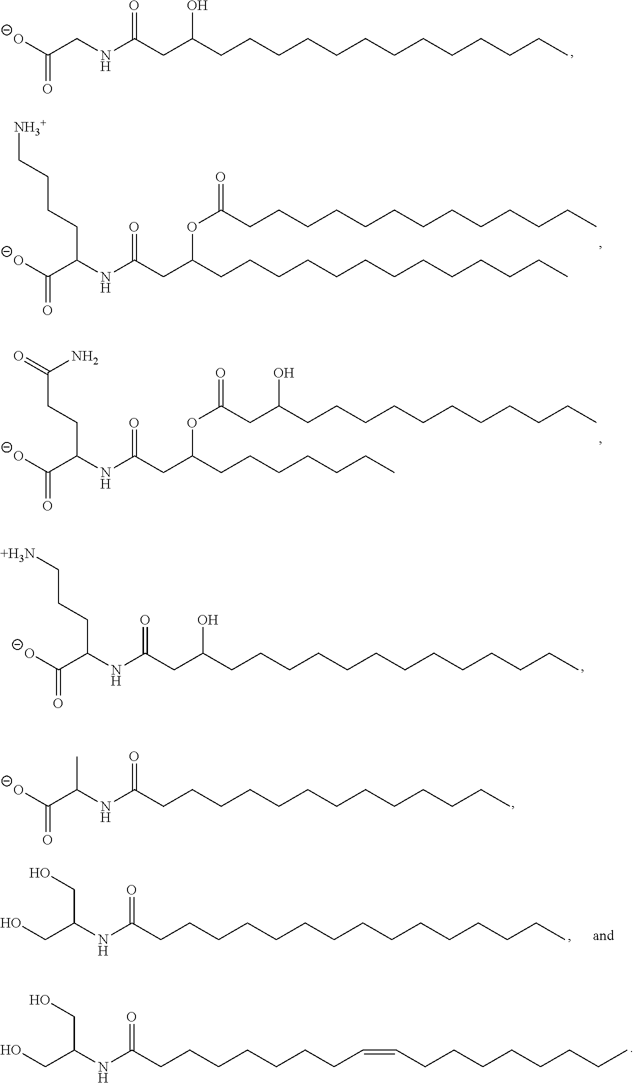

53. The method of claim 48, wherein the N-acyl amide is N-oleoyl serinol.

54. The method of claim 42, wherein the composition is administered in a therapeutically effective amount.

55. The method of claim 42, wherein the composition further comprises a pharmaceutically acceptable carrier, diluent, buffer, or excipient.

56. The method of claim 42, wherein the adenocarcinoma is found in the digestive system of the subject.

57. The method of claim 42, wherein the adenocarcinoma is found in the liver, pancreas, small intestine, large intestine, colon, or stomach.

58. The method of claim 42, wherein the adenocarcinoma is hepatocellular carcinoma.
